(19) United States
(12) Patent Application Publication
(10) Pub. No.: US 2026/0207673 A1
(43) Pub. Date: Jul. 23, 2026

(54) MICROGLIA-LIKE CELLS

(71) Applicant: SYDDANSK UNIVERSITET, Odense M (DK)

(72) Inventor: Agnieszka WLODARCZYK, Odense M (DK)

(73) Assignee: SYDDANSK UNIVERSITET, Odense M (DK)

(21) Appl. No.: 19/139,052

(22) PCT Filed: Dec. 15, 2023
(86) PCT No.: PCT/EP2023/086057

§ 371 Date: Jun. 13, 2025

(30) Foreign Application Priority Data
Dec. 15, 2022 (EP) 22213790.3

(51) Int. Cl. A61K 35/30; A61P 25/28; C12N 5/079

(52) U.S. Cl. CPC A61K 35/30; A61P 25/28; C12N 5/0622; C12N 2501/105; C12N 2501/155

(57) ABSTRACT

The present invention relates to the provision of novel sources of microglia-like cells having a unique phenotype. In particular, the present invention relates to medical uses of such microglia-like cells, in particular in relation to treatment of neurodegenerative diseases.

### TECHNICAL FIELD OF THE INVENTION

The present invention relates to the provision of novel microglia-like cells having a unique phenotype. In here is also provided sources of such microglia-like cells, and media conditioned by such microglia-like cells. In particular, the present invention relates to medical uses of such microglia-like cells and the conditioned media, in particular in relation to treatment of neurological diseases, such as neurodegenerative diseases.

### BACKGROUND OF THE INVENTION

Global human population is aging rapidly with increasing life expectancy. As ageing is the primary risk for neurodegenerative diseases including Alzheimer's Disease (AD), progressive form of multiple sclerosis and ischemic stroke, they are becoming one of the biggest medical issues of our times (1). For instance, AD is the most common form of dementia, and the disease incidence are continuously rising, expected to double within the next 20 years (2). Despite decades of research there are only few drugs for AD—none of which can stop the progression of the disease (3) and there are no treatments that can reverse disabilities in other neurological diseases. Thus, finding a way to induce neuro-regeneration has become an urgent enterprise.

A hallmark of many neurodegenerative diseases is an activation of microglia—the resident macrophages in the brain, concentrated around amyloid beta plaques (4) in AD, demyelinated plaques in MS and around the infarcts in Stroke.

Microglia are yolk-sac derived CNS-resident immune cells. They are often referred to as brain macrophages and indeed share many phenotypic features with macrophages, especially when activated in response to pathological events. However, it is now recognized that they are of a separate lineage. Classically, microglia are patrolling the CNS clearing up debris and reacting rapidly for danger signals and inducing reaper mechanisms, which makes them important for CNS homeostasis (5). Over the last decade microglia emerged as a critical player in neurodevelopment. They are essential in a process of synaptic pruning by phagocytizing weak synapses during neurodevelopment and learning processes (6-8), express factors involved in maturation of astrocytes, oligodendrocytes and neurons (9), induce primary myelination (9, 10) most likely by stimulation myelinogenic program in oligodendrocytes (11) and support survival of layer V cortical neurons during postnatal development by producing neuroprotective insulin-like growth factor 1 (IGF1) (12). Interestingly, microglia lose these neurogenic properties during maturation (9). Microglia are long-lived cells that are not replaced from blood derived precursors but proliferate instead to sustain their rather stable number (13). Thus, over the years they are exposed to many activating cues, as well as accumulate somatic mutations during divisions, which all lead to dramatic phenotypical changes (14). This in turn underlies the dysregulation in microglia that can no longer efficiently fulfill their functions leading to progressing neurodegeneration and cognitive decline.

US 2022/241344 A1 concerns a microglia progenitor cell derived from bone marrow and methods for their isolation; as well as use of said cells for therapy of disorders of the CNS.

Wlodarczyk et al. (The EMBO Journal. Vol 36, NO 22, 2017) discloses that a novel microglial subset plays a key role in myelinogenesis in developing brain.

Benmamar-Badel (Frontiers in Immunology, April 2020, Volume 11, Article 430) reviews protective microglial subset in development, aging and disease.

WO 2018/071898 A1 provides compositions and methods for the treatment or prevention of a neurological disease or disorder of the central nervous system (e.g., a storage disorder, lysosomal storage disorder, neurodegenerative disease, etc.) by reconstitution of brain myeloid cell and microglia upon transplantation of hematopoietic cells enriched in microglia reconstitution potential.

Hence, new sources of “microglia-like cells” would be advantageous, and in particular a more efficient and/or reliable treatment of neurodegenerative diseases would be advantageous.

### SUMMARY OF THE INVENTION

In here is provided a cell-based therapy that aims at replacing/supplementing old and exhausted microglia with young and neurosupportive neonatal microglia-like cells. Thus, the present invention relates to the provision of novel microglia-like cells having a unique phenotype. In here is also provided sources of such microglia-like cells. In particular, the present invention relates to medical uses of such microglia-like cells, in particular in relation to treatment of neurological diseases, such as neurodegenerative diseases and cardiovascular diseases such as stroke.

Examples 1 and 2 show that neonatal microglia-like cells have therapeutical potential against MS and age-related cognitive decline respectively. Example 1 further indicates that CD11c⁺ neonatal microglia have higher neuroprotective capacity than their CD11c⁻ counterparts.

Example 3 shows that primitive macrophages (microglia-like cells) can be isolated from umbilical cord and placenta of mice. These cells closely resemble yolk sac-derived microglia progenitors.

Example 4 shows that primitive macrophages (microglia-like cells) can be isolated from umbilical cord and placenta of humans. These cells closely resemble yolk sac-derived microglia progenitors.

Example 5 shows that transplantation of umbilical cord macrophages (UCM) (neonatal microglia-like cells) can improve short-and long-term memory in animal model of Alzheimer's Disease (AD) and reduce amyloid beta plaques. hUCM show phagocytic capacity towards amyoid beta.

Example 6 shows that transplantation of UCM leads to functional improvement in animal model of stroke and that hUCM have strong pro-angiogenic properties.

Example 7 shows that conditioned medium can be generated from neoMG, likely comprising osteopontin and/or IGF1. The conditioned medium is capable of enhancing learning abilities and memory in aged animals.

Thus, an object of the present invention relates to the provision of novel sources of neonatal microglia-like cells.

In particular, it is an object of the present invention to provide improved treatments of neurodegenerative diseases.

Thus, one aspect of the invention relates to a composition comprising one or more microglia-like cells, such as neonatal microglia-like cells, wherein the one or more microglia-like cells has the phenotype CX3CR1+; CD45+, and cKit−. Preferably, the cells are also CD14− and/or CD11b+.

Another aspect of the present invention relates to a process for providing tissue-resident macrophages (TRM), the process comprising
a) providing umbilical cord tissue and/or placenta tissue;
b) purifying TRMs from said umbilical cord tissue and/or placenta tissue by selecting for cells that are CX3CR1+; and
c) providing macrophages which are CX3CR1+.

Yet another aspect of the present invention is to provide cells obtained by or obtainable by a process according to the invention.

A further aspect of the present invention is provided a process of providing a conditioned medium, the process comprising the steps:
a) providing at least one cell as defined herein;
b) contacting a media with the at least one cell for a time sufficient to condition the media, such as for at least 12 hours, such at for at least 24 hours;
c) removing the at least one cell from the media, thereby providing a conditioned media; and
d) optionally, diluting or concentrating the provided conditioned media.

An additional aspect relates to a conditioned media obtained by or obtainable by the process as described above.

Still, an aspect of the invention relates to the composition according to the invention or cells according to the invention, or conditioned medium according to the invention, for use as a medicament.

In a related aspect, the invention relates to the composition according to the invention or cells according to the invention, or conditioned medium according to the invention for use in the treatment, alleviation and/or prevention of neurodegenerative diseases, demyelinating conditions, stroke and age-related cognitive decline.

Yet an aspect of the invention relates to an in vitro method for determining the effect of at least one agent on a cell as defined according to the invention, the method comprising
a) providing at least one cell as defined according to the invention;
b) contacting the at least one cell from step a) with the biological agent; and
c) determining the effect of the agent on the least one cell.

### BRIEF DESCRIPTION OF THE FIGURES

FIG. 1

FIG. 1 shows that transplantation of neonatal microglia ameliorates EAE. EAE progression in mice with symptomatic EAE (grade 2), which received: PBS, 2×10⁵ MACS-sorted adult or neonatal microglia by injection into cisterna magna. A few mice reached the ethical end point ( [symbol] ) and had to be sacrificed before the end of experiment. A) Data pooled from three individual experiments are presented as means±SEM (n ≥7) *p<0.05, **p<0.01 assessed using two-way ANOVA. B) Representative micrographs of three individual experiments showing demyelination and CD45+leukocyte infiltration in mice with EAE that received PBS or were transplanted with adult or neonatal microglia. C) Analysis of progression of demyelination presented as percentage of demyelinated spinal cord at d0, d2, d3, d4, d5 post transplantation. D) EAE progression and E) maximum EAE grade in mice with symptomatic EAE (grade 2) that received 5×10⁴ FACS-sorted neonatal CD11c+ or CD11c− microglia by injection into cisterna magna or were left untreated Data pooled from two individual experiments are presented as means±SEM (n≥3)*p<0.05, **p<0.01 assessed using two-way ANOVA (D) or two-tailed Mann-Whitney U-test (E). F) Data pooled from ten individual experiments are presented as means±SEM (n=27, 16, 30) **p<0.01, ***p<0.001, ****p<0.0001 assessed using two-way ANOVA.

FIG. 2

FIG. 2 shows that transplantation of neonatal microglia improves short- and long-term memory in aged animals. Short- (4 days) and long-term (11 days) memory assessment in aged animals that received 3 constitutive injections of PBS (white) or 2×10⁵ MACS-sorted neonatal (black) microglia into cisterna magna compared to unmanipulated young (12 weeks old) animals measured as A) primary latency and B) primary error holes. Data pooled from three individual experiments are presented as means±SEM (n≥3) *p<0.05, **p<0.01 assessed using one-way ANOVA (A). C) Number of amyloid beta plaques in 7 month old 5xFAD animals that received 2×10⁵ MACS-sorted neonatal microglia or PBS into cisterna magna n=2. D) and E) as A) and B), however with data pooled from four individual experiments, presented as means±SEM (n≥6).

FIG. 3

FIG. 3 shows pregnancy associated tissues as a source for microglia progenitors. A) Representative flow cytometry profiles of yolk sac, umbilical cord and placenta showing identification of primitive macrophages (CD45+, CX3CR1+, c-kit−, Ly6C−). B) Representative micrographs showing colonization of corpus callosum of otherwise unmanipulated mice intra-striatally transplanted with 2×10⁵ UCM acquiring IBA1 expression.

FIG. 4

FIG. 4 shows identification of primitive macrophages in human umbilical cord and placenta. Representative micrographs of A) umbilical cord and B) placenta showing CX3CR1+ primitive macrophages.

FIG. 5

FIG. 5 shows that transplantation of UCM improves short-and long-term memory in animal model of AD. Short- (4 days) and long-term (11 days) memory assessment in microglia depleted 7 months old 5xFAD animals aged animals that received injections of PBS (white) or A) 2×10⁵ MACS-sorted UCM via ICV; B) 2×10⁵ MACS-sorted UCM+50 ng MCSF via ICV followed by 50 ng MCSF i.t; or C) 50 ng MCSF ICV followed by 2×10⁵ MACS-sorted UCM i.t, and compared to wild type littermates.

Top picture in A-C: Mean primary latency(s). Bottom picture in A-C: Primary error holes (n).

FIG. 6

Short- (4 days) and long-term (11 days) memory assessment in microglia depleted 7 months old 5xFAD animals aged animals that received injections of PBS (white) or A) 2×10⁵ MACS-sorted UCM via ICV B) 2×10⁵ MACS-sorted UCM+50 ng MCSF via ICV followed by 50 ng MCSF i.t and compared to wild type littermates; Data pooled from four individual experiments are presented as means±SEM (n=16, 16, 5) *p<0.05, assessed using two-way ANOVA. C) Number of amyloid beta plaques in 7 month old 5xFAD animals that received 2×10⁵ MACS-sorted UCM+50 ng MCSF via ICV followed by 50 ng MCSF i.t a or PBS into cisterna magna n=4. Data pooled from two individual experiments are presented as means±SEM (n=4) *p<0.05, assessed using student's t-test.

Top picture in A-B: Mean primary latency (s). bottom picture in A-B: primary error holes (n).

FIG. 7

FIG. 7 shows That UCM isolated from human UC phagocytose amyloid beta in vitro. Representative micrograph of three individual experiments showing hUCM phagocytosing AF488− Beta-Amyloid (1-42) after 24 h in vitro culture.

FIG. 8

FIG. 8 shows that transplantation of UCM improves functional outcomes in ischemic stroke model.

Grip strength analysis (5 days survival) normalised to the baseline in animals transplanted i.t 2×10⁵ MACS-sorted UCM or PBS 24 h after permanent middle cerebral artery occlusion. Data from one experiment (n=5), *p<0.05, assessed using one-way ANOVA.

FIG. 9

FIG. 8 shows pro-angiogenic properties of UCM isolated from human UC in vitro. A), B) Representative micrographs from four individual experiments (4 different donors) and C) bar graph showing increased number endothelial tubes formed by HUVEC cells in vitro upon stimulation with hUCM conditioned media in comparison to control media. *p<0.05, assessed using mann whitney test.

FIG. 10

FIG. 10 shows i.t injected neo-microglia (neoMG) predominantly localizes in the meninges and, to a lesser extent, in the cerebellum in aged animals.

FIG. 11

FIG. 11 shows conditioned media from neoMG significantly enhances learning abilities and memory, with a particularly notable improvement observed in long-term memory in aged animals.

Short- (4 days) and long-term (11 days) memory assessment in aged animals that received 3 constitutive injections of ctr media (white) or conditioned media (black) into cisterna measured as A) mean primary latency during training; B) primary latency and C) primary error holes. Data pooled from two individual experiments are presented as means±SEM (n≥5) *p<0.05, **p<0.01 assessed using one-way ANOVA

The present invention will now be described in more detail in the following.

### DETAILED DESCRIPTION OF THE INVENTION

### Definitions

Prior to discussing the present invention in further details, the following terms and conventions will first be defined:

### Microglia

Microglia are the tissue-resident macrophages of the central nervous parenchyma. In mammals, microglia are thought to originate from yolk sac precursors and posteriorly maintained through the entire life of the organism.

### Pre-Microglia Cells/Microglia-Like Cells/Microglia Progenitor Cells/Neonatal Microglia-Like Cells

In the present context, the terms “pre-microglia cells”, “microglia-like cells”, “microglia progenitor cells” and “neonatal microglia-like cells” are cells capable of engrafting the mature brain mainly under pathological conditions and/or mediate their effect being in the Cerebrospinal fluid (CSF) and not going into the parenchyma. These cells implement great parts of the microglial phenotype, but they might not completely adopt the “true microglia” features. These terms may be used interchangeable throughout this document.

Preferably the cells are tissue derived macrophages (derived from tissue-resident macrophages (TRM)), such as being derived from the placenta or umbilical tissue. Thus, the cells may be derived from a source different from the yolk sac and the central nervous parenchyma.

For humans, the “pre-microglia cells”/“microglia-like cells”/“neonatal microglia-like cells”/“neonatal microglia-like cells” preferably have the phenotype CX3CR1+; CD45+; CD11b+, cKit− and CD14+/CD14−. More preferably the cells have the phenotype CX3CR1+; CD45+; cKit−. More preferably the cells are CD14−.

### CX3CR1

CX3CR1 is also known as “V28”, “CCRL1”, “GPR13” “CMKDR1”, “GPRV28 and “CMKBRL1”. The protein encoded by CX3CR1 is a receptor for fractalkine, which is a transmembrane protein and chemokine involved in the adhesion and migration of leukocytes.

### CD45 (PTPRC, LCA, LY5)

CD45 is a receptor linked protein tyrosine phosphatase present in all cells of the hematopoietic lineage except erythrocytes and plasma cells. Originally known as common leukocyte antigen or protein tyrosine phosphatase receptor type C it was given the designation CD45 by the HCDM. CD45 is highly conserved across mammals as well as having homologs in several more ancient species. Its expression is strong among all hematopoietic cells.

### Ly6C

In mice the Ly6C antigen is expressed on monocytes, macrophage/dendritic cell precursors in mid-stage development (late CFU-M, monoblasts and immature monocytes), granulocytes, and on a wide range of endothelial cells and subpopulations of B- and T-lymphocytes. It is routinely used to discriminate monocytes/macrophages from other leukocytes. The Ly6C antigen does not exist in human.

### CD14

CD14 the protein is a surface antigen that (similarly to Ly6C in mouse) is preferentially expressed on monocytes/macrophages.

cKIT (C-Kit, CD117, MASTC, PBT, SCFR, KIT)

cKit (CD117) is expressed by some haematopoietic stem cells (approx. 70% of CD34+ve cells in normal bone marrow are CD117+ve), some acute myeloid leukaemias and by mast cells. Functionally CD117 is a receptor for stem cell factor, and has receptor tyrosine kinase activity.

### CD11B, ITGAM, CR3A, MAC-1

Integrin ITGAM/ITGB2 is implicated in various adhesive interactions of monocytes, macrophages and granulocytes as well as in mediating the uptake of complement-coated particles and pathogens 1, 2. It is identical with CR-3, the receptor for the iC3b fragment of the third complement component. It probably recognizes the R-G-D peptide in C3b. Integrin ITGAM/ITGB2 is also a receptor for fibrinogen, factor X and ICAM1.

### MCSF

In the present context, the terms “MCSF” or “macrophage colony-stimulating factor” or “M-CSF” or “colony stimulating factor 1” or “CSF1” relate to the secreted cytokine which e.g. causes hematopoietic stem cells to differentiate into macrophages or other related cell types.

### CD47

CD47 (Cluster of Differentiation 47) also known as integrin associated protein (IAP) is a transmembrane protein that in humans is encoded by the CD47 gene. CD47 belongs to the immunoglobulin superfamily and binds the ligands thrombospondin-1 (TSP-1) and signal-regulatory protein alpha (SIRPa). CD-47 acts as a don't eat me signal to macrophages.

### HLA-E

HLA-E (HLA class I histocompatibility antigen, alpha chain E) also known as MHC class I antigen E is a protein that in humans is encoded by the HLA-E gene. The human HLA-E is a non-classical MHC class I molecule that is characterized by a limited polymorphism. Inhibits NK-cell cytotoxicity.

### Composition

As outlined above and in the example section, the inventing team has identified a source of microglia-like cells in umbilical cord tissue and the placenta. These cells have a unique phenotype. Thus, a first aspect of the invention relates to a composition comprising one or more microglia-like cells, such as neonatal microglia-like cells, wherein the one or more microglia-like cells has the phenotype CX3CR1+; CD45+, CD11b+, and cKit−. Preferably, the cells are also CD14−.

Another aspect of the invention relates to composition comprising one or more microglia-like cells, such as neonatal microglia-like cells, wherein the one or more microglia-like cells has the phenotype CX3CR1+; CD45+, cKit−, CD47+ CD11b+/CD11b−, CD14+/CD14−. Preferably, the cells are also CD11b+.

A further aspect of the invention relates to composition comprising one or more microglia-like cells, such as neonatal microglia-like cells, wherein the one or more microglia-like cells has the phenotype CX3CR1+; CD45+, and cKit−. Such a composition may preferably also display one or more of the expression patterns CD47+, CD11b+ or CD11b−, and/or CD14+ or CD14−. Preferably, the cells are also CD11b+. In one embodiment of the present disclosure the cells are also HLA-E+.

Interestingly, in addition to the markers that can easily be shown on viable cells, such as in flow cytometry, the cells of the invention also appear to express osteopontin and/or insulin-like growth factor (IGF1) (example 7). In contradiction to the other markers, these proteins are however secreted. Thus, in an embodiment of the invention, the cells are also secreting osteopontin and/or IGF1. Whether the cells secret such compounds may be more laborious to understand on viable cells, however when investigating whether a composition of cells express these proteins, a portion thereof may be taken and investigated in more detail. In one embodiment of the present disclosure the cells are secreting or expressing osteopontin. In one embodiment of the present disclosure the cells are secreting or expressing IGF1. In one embodiment of the present disclosure the cells are secreting or expressing osteopontin and IGF1. However, in other embodiments of the invention, the genes of osteopontin and IGF1 are used to identify whether the cells express osteopontin and/or IGF1, thus the skilled person would be looking for SPP1 and/or IGF1 expression.

As shown in examples 1-3 and 5 such cells have a therapeutic potential in mice models. In example 4, it is shown that a corresponding cell type can be identified in human umbilical cord tissue and the placenta.

In an alternative aspect, the invention relates to a composition comprising one or more cells, wherein the one or more cells has the phenotype CX3CR1+; CD45+, CD11b+, and cKit−. It is to be understood that the aspects and embodiments relating to compositions comprising “microglia-like cells” are also applicable to this aspect, where the cells are not defined as “microglia-like cells”. In an embodiment, however, the cells are “microglia-like cells”.

In another embodiment, the microglia-like cells are also CD11C+. Example 1 shows that CD11c⁺ neonatal microglia have higher neuroprotective capacity than their CD11c⁻ counterparts. Further, as shown in example 3, transcriptomes of the fetal cluster 7 and maternal cluster 4 significantly overlapped with CD11c+ neonatal microglia signature.

As outlined in the example section the microglia-like cells can be isolated from tissue. Thus, in an embodiment, the microglia-like cells are tissue-resident macrophages (TRM).

In another embodiment, the microglia-like cells are derived from umbilical cord tissue. Thus, in a related embodiment, the microglia-like cells are derived from umbilical cord tissue, different from umbilical cord blood (or free from cord blood).

In a further embodiment, the microglia-like cells are derived from placenta tissue. In a related embodiment, the microglia-like cells are derived from placenta tissue, different from umbilical cord blood (or free from cord blood).

As also outlined throughout the example section the cells are considered capable of functioning as a medicament in the brain. Thus, in an embodiment, the microglia-like cells are cells capable of engrafting the mature brain, such as under pathological conditions.

In yet another embodiment, the microglia-like cells have the phenotype of microglia.

As shown in example 4, microglia-like cells have been identified in both placenta and umbilical cord tissue. Thus, an aspect of the invention relates to an (isolated) composition comprising microglia-like cells isolated from umbilical cord tissue.

Another aspect of the invention relates to a (isolated) composition comprising microglia-like cells isolated from placenta tissue.

In an embodiment, the microglia-like cells have the phenotype CX3CR1+, CD11b+, CD45+, and cKit−. In yet an embodiment, the microglia-like cells are further CD14−.

In another embodiment, the microglia-like cells are also CD11C+.

In an embodiment, the composition comprises one or pharmaceutical acceptable carriers or diluents and/or being a pharmaceutical composition.

Cytokines, such as MCSF, may improve the effect of the microglia like cells, when used as a medicament. Thus, in an embodiment, the composition further comprises one or more cytokines, such as MCSF and/or IL34, preferably MCSF. In the example section tests both with and without MCSF have been carried out.

In yet an embodiment, the cells are stimulated with one or more cytokines in vitro, before use as a medicament.

In an embodiment, the microglia-like cells are derived from a mammal, preferably a primate, and more preferably a human.

In an embodiment, the composition comprises in the range 10⁴-10⁸ of said microglia-like cells, such as 10⁵-10⁸, such as 10⁶-10⁷, or 10⁷-10⁸.

### Process for Preparing Microglia-Like Cells from Tissue

The present invention also relates to a process for purifying cells, such as cells according to the present invention. Thus, an aspect of the invention relates to a process for providing tissue-resident macrophages (TRM), the process comprising
a) providing umbilical cord tissue and/or placenta tissue;
b) purifying TRMs from said umbilical cord tissue and/or placenta tissue by selecting for cells that are CX3CR1+; and
c) providing macrophages which are CX3CR1+.

As shown in example 3, cells can be isolated using only CX3CR1 as a selective marker.

### Step a)

In a preferred embodiment, the tissue is provided (e.g. being previously obtained from a subject) from a human subject. In yet a preferred embodiment, the provided umbilical cord tissue or placenta tissue is rinsed to remove blood.

### Step B)

The purification step b) can be carried out using different methods. Thus, in an embodiment, in purification step b), purification is performed by cell sorting such as FACS or Magnetic-activated cell sorting (MACS).

Further selection markers (either positive or negative selection) can be included. Thus, in an embodiment, in step b), the cells are further selected for being CD45+, and/or CD14−, and/or cKit−.

In yet an embodiment, in step b), the cells are further selected for being CD11B+ and/or CD11C+.

### Step c)

In an embodiment, in step c) the provided cells are CX3CR1+, CD11b+, CD45+, and cKit−. In a related embodiment, the provided cells are further CD14−.

In a related embodiment, in step c) the provided cells are CD11C+.

In a preferred embodiment, the provided cells are cells as defined according to the present invention.

### Step d)

The provided cells may be stored for later use. Thus, in an embodiment, the process further comprises the step d) of cryopreserving the cells.

### Conditioned Media

As shown by example 7, a media conditioned by the cells described herein, have therapeutic uses. Thus in a further aspect of the present invention is provided a process of providing a conditioned medium, the process comprising the steps:
a) providing at least one cell as defined herein;
b) contacting a media with the at least one cell for a time sufficient to condition the media, such as for at least 12 hours, such at for at least 24 hours;
c) removing the at least one cell from the media, thereby providing a conditioned media; and
d) optionally, diluting or concentrating the provided conditioned media.

A related aspect is thus a process of providing a conditioned media, the process comprising the steps:
a) providing one or more microglia-like cells, such as neonatal microglia-like cells, wherein the one or more microglia-like cells has the phenotype CX3CR1+, CD45+, and cKit− optionally, wherein the microglia-like cells are also CD11b+;
b) contacting a media with the one or more microglia-like cells for a time sufficient to condition the media, such as for at least 24 hours;
c) removing the one or more microglia-like cells from the media, thereby providing a conditioned media; and
d) optionally diluting or concentrating the provided conditioned media.

Since the use of the media is very important and has therapeutic implications, the application also provides for the conditioned media from such cells, and the media that is derived from such processes.

Thus, an aspect relates to a conditioned media obtained by or obtainable by the process as described above.

The media may be any related growth culture media, such as Dulbecco's Modified Eagle Medium. In specific embodiments, the media may be supplemented with one or more components selected from the group consisting of: Nutrient Mixture F12, FBS, penicillin-streptomycin, N-2, interleukin 34 (IL34), and mL macrophage colony-stimulating factor (MCSF). However, preferably for using the conditioned media for therapy, a xenofree media is used.

Thus, another aspect of the invention relates to a conditioned medium comprising secreted molecules, such as proteins, from one or more microglia-like cells, such as neonatal microglia-like cells, wherein the one or more microglia-like cells has the phenotype CX3CR1+, CD45+, and cKit-optionally, wherein the microglia-like cells are also CD11b. In an embodiment of the invention, the conditioned medium is comprising osteopontin and/or IGF1. In one embodiment of the present disclosure the conditioned medium is comprising osteopontin. In one embodiment of the present disclosure the conditioned medium is comprising IGF1. In one embodiment of the present disclosure the conditioned medium is comprising osteopontin and IGF1. In one embodiment of the present disclosure the conditioned media comprises one or more proteins selected from the group consisting of osteopontin and/or IGF1.

In one embodiment of the present disclosure, the conditioned medium is capable of improving learning capabilities and/or restoring long-term memory, such as in aged animals.

### Product by Process

As outlined in the example section, the isolated cells have a unique phenotype and are able to function as medicaments, e.g. in the treatment of neurological diseases. Hence, an aspect of the invention relates cells obtained by or obtainable by a process according to the invention.

### Medical Uses

As outlined above, the cells can be used in the treatment of different diseases.

Thus, an aspect of the invention relates to the composition according to the invention or cells according to the invention, for use as a medicament. Examples 1, 2 and 5 discloses the potential medical uses of the compositions and cells according to the invention. Example 6 shows a further medical use according to the invention. Contrary to many known treatment regimens, the present invention aims at replacing and/or supplementing/supporting old and exhausted microglia with young and neurosupportive neonatal microglia-like cells. Thus, it is not necessarily a goal of the present invention to provide cells that should remain in the brains, however a goal is also to provide cells capable of supporting the resident microglia. This is for instance seen by Example 7, showing that cells injected for therapy localizes in the meninges, and further shows that conditioned media also provides a restorative effect.

In a related aspect, the invention relates to the composition according to the invention or cells according to the invention, for use in the treatment, alleviation and/or prevention of neurodegenerative diseases, demyelinating conditions, stroke and age-related cognitive decline.

In a related aspect, the invention relates to the conditioned medium according to the invention, for use in the treatment, alleviation and/or prevention of neurodegenerative diseases, demyelinating conditions, stroke and age-related cognitive decline. In a preferred embodiment the conditioned medium is for use in the treatment alleviation and/or prevention of neurodegenerative diseases and/or age-related cognitive decline, preferably age-related cognitive decline. That the conditioned medium can be used as a therapeutic is for instance shown in example 7, without being bound by theory, the conditioned medium can be used for the same therapies as the cells themselves.

In an embodiment, the neurodegenerative disease is selected from the group consisting of Alzheimer′ Disease, Parkinson disease, multiple sclerosis (MS), and amyotrophic lateral sclerosis (ALS). Example 1 shows neonatal microglia transplantation leads to functional improvement in an animal model of MS. Further, Example 5 demonstrates the therapeutic potential of umbilical cord macrophages (UCM) in Alzheimer's Disease (AD).

In another embodiment, the demyelinating condition is selected from the group consisting of leukodystrophies, multiple sclerosis, spinal cord injury, peripheral nerve damage, Parkinson's disease, amyotrophic lateral sclerosis (ALS), and Alzheimer's disease. In a preferred embodiment, multiple sclerosis is the demyelinating diseases.

In a further embodiment, stroke is selected from the group consisting of ischemic stroke and hemorrhagic stroke. In a further embodiment, stroke is selected from the group of cardiovascular diseases consisting of Coronary artery disease, peripheral artery disease, cerebrovascular disease incl. ischemic stroke and hemorrhagic stroke, and aortic atherosclerosis. In a further embodiment, cardiovascular diseases are selected from the group consisting of Coronary artery disease, peripheral artery disease, cerebrovascular disease, ischemic stroke and hemorrhagic stroke. Example 6 shows that transplantation of UCM leads to functional improvement in animal model of stroke and that hUCM have strong pro-angiogenic properties.

In yet a further embodiment, age related cognitive decline is selected from the group consisting of decline of memory, such as decline of short-term memory, decline of long-term memory, and/or decline of spatial memory. Example 2 shows that transplantation of neonatal microglia leads to improvement of short-term memory and complete restoration of long-term memory. Hence, neonatal microglia transplantation leads to a restoration of the age-related memory loss.

The composition and cells according to the invention may be administered by different administration routes. Thus, in an embodiment, the composition or cells according to the invention is administered by intrathecal delivery, such as intracisternal delivery, intracerebroventricular delivery, intracerebral delivery, intranasal delivery or by injection/delivery into the cisterna magna. As shown in e.g. example 5 cells may be administered by intracerebroventricular delivery.

The treatment may be improved by the addition of cytokines such as MCSF and/or CSF1R agonists. Thus, in an embodiment, the composition or cells are administered before and/or after and/or simultaneously to administration of cytokines, such as such as MCSF and/or IL34, preferably MCSF or CSF1R agonists. As shown in e.g. example 5, MCSF administration may improve the treatment.

In a preferred embodiment, the treatment is provided to a mammal, preferably a human subject.

In an embodiment, said subject has been subjected to microglia depletion before said treatment. Microglia depletion can be performed by different means known to the person skilled in the art, such as clodronate liposomes, genetic models and CSF1R inhibitors. Microglial depletion provides an empty niche that stimulates production of new microglia and/or allows for repopulation with the cells according to the present invention.

### An In Vitro Method for Determining the Effect of an Agent on the Cells

The cells and compositions according to the invention may also find use in vitro. Thus, an aspect of the invention relates to an in vitro method for determining the effect of at least one agent on a cell as defined according to the invention, the method comprising
a) providing at least one cell as defined according to the invention;
b) contacting the at least one cell from step a) with the biological agent; and
c) determining the effect of the agent on the least one cell.

In an embodiment, the agent is selected from the group consisting of growth factors, trophic factors, growth factor receptors, neurotransmitters, neuropeptides, neurotrophic factors, hormones, enzymes, cytokines, lymphokines, anti-angiogenic factors, transcription factors, proliferation factors and antibodies.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Therapeutic Potential of Neonatal Microglia in MS

### Aim of Study

To demonstrate therapeutic potential of neonatal microglia in MS.

### Materials and Methods

### Mice

C57BL/6j bom female mice aged 7-8 weeks were obtained from Taconic Europe A/S, and maintained as a breeding colony in the Biomedical Laboratory, University of Southern Denmark (Odense). Neonatal C57BL/6j bom mice (p3-p6) used for experiments were of mixed sex.

### EAE-Model

Seven to ten weeks old female mice were immunized by injecting subcutaneously 100 μl of an emulsion containing 300 μg of myelin oligodendrocyte glycoprotein (MOG)p35-55 (TAG Copenhagen A/S, Frederiksberg, Denmark) in incomplete Freunds adjuvant (DIFCO, Albertslund, Denmark) supplemented with 400 μg H37Ra Mycobacterium tuberculosis (DIFCO). Bordetella pertussis toxin (300 ng; Sigma-Aldrich, Brøndby, Denmark) in 200 μl of PBS was injected intraperitoneally at day 0 and day 2. Animals were monitored daily from day 5 and scored on a 6-point scale as follows: 0, no symptoms; 1, partial loss of tail tonus; 2, complete loss of tail tonus; 3, difficulty walking, 4, paresis in both hind legs; 5, paralysis in both hind legs; 6, front limb weakness; Due to ethical consideration mice were sacrificed when they reached grade 6 or 24 hours after hind legs paralysis.

### CNS Dissociation:

To isolate neonatal microglia from brain, PN3-6 mice were terminally anaesthetized with 200 mg/kg of pentobarbital and intracardially perfused with ice-cold PBS. Brain tissue was collected, and single cell suspensions were generated using either enzymatic dissociation (total neonatal microglia) with Neural Tissue Dissociation Kit (P) (Miltenyi Biotec) or mechanical dissociation by forcing through a 70 mm cell strainer (BD Biosciences) (CD11c+ neonatal microglia); dissociations were followed by 37% Percoll (GE Healthcare Bio-sciences AB) gradient centrifugation.

### Magnetic-Activated Cell Sorting (MACS-Sorting)

Cell suspension was incubated for 15 min with CD11b MicroBeads (Miltenyi Biotec), washed and sorted on manual MACS separator (Miltenyi Biotec) using MS column (Miltenyi Biotec). To ensure high purity, the eluted cells were put through second MS column.

### Fluorescencet-Activated Cell Sorting (FACS-Sorting)

Cell suspension was incubated in blocking buffer (2% fetal bovine serum, Merck Life Science A/S, Søborg, Denmark; anti-Fc receptor CD16/CD32, BD Pharmingen; and 50 μg/mL Syrian Hamster IgG, Jackson ImmunoResearch Laboratories Inc., West Grove, PA, USA) before staining. Staining was performed using anti-CD45 (Clone 30-F11; Biolegend), anti-CD11b (Biolegend) and biotin conjugated anti-CD11c (BD Pharminogen) antibodies in PBS, 1% FBS and finally with streptavidin-APC (Biolegend) antibodies and live/dead marker (Sytox Blue Dead Cell Stain; Thermo Fisher Scientific). Cells were sorted on a FACS Aria III (BD Biosciences).

### Neonatal Microglia Transplantation

Total microglia from 3-6 days old and adult CNS were isolated by MACS-sorting (purity≥95%). Mice with grade 2 of EAE were randomized into three groups that received 10 μl PBS, 2×10⁵ neonatal or adult microglia in PBS by injection into cisterna magna. Alternatively, CD11c+ and CD11c− neonatal microglia were isolated by FACS from 3-5 days old CNS. Mice that reached grade 2 of EAE were randomized into three groups that received 5×10⁴ of CD11c⁺ or CD11c⁻ neonatal microglia in PBS by injection into cisterna magna or were left untreated. Mice were further monitored daily by a blinded investigator for 4 days from cell transplantation.

In order to localize the transplanted microglia within the recipient SC lesions, cells were PKH26 (Sigma Aldrich) labeled (according to manufacturer's protocol) prior to transplantation. Recipient mice were sacrificed 2 days after cell transplantation.

### Histology

Twelve-micrometer sections from 4% PFA-fixed, frozen spinal cords of perfused mice were cut on a cryostat and stored in de Olmos cryoprotectant solution (17) at −14° C. or in −20° C. on Superfrost Plus slides (Thermo Scientific) respectively. Sections were incubated for 30 min with 10% methanol, 10% H₂O₂ in PBS to fix and block endogenous peroxidase. After repeated rinses with 0.2% Triton X100 in PBS (PBST) they were incubated for 1 h in 3% BSA in PBS to block unspecific binding. Next, sections were incubated overnight at 4° C. with biot-anti-MOG (purified by Protein G affinity from supernatant of the Z2 Hybridoma kindly provided by Christopher Linington, Glasgow, Linington lab). Following primary antibody incubation, the sections were washed with PBST and incubated for 1 h with strept-HRP. Following secondary antibody incubation, the sections were washed with PBS and incubated for 8 min with DAB solution and analyzed by ImageJ software.

Alternatively, sections were washed in PBS and incubated for 10 min in ice-cold acetone. After repeated rinses with 0.2% Triton X100 in PBS (PBST) they were incubated for 1 h in 3% BSA in PBS to block unspecific binding. Next, sections were incubated with anti-CD45 PE (biolegend) for 1 h room temperature (RT). After immunofluorescent labeling, nuclei were visualized by DAPI staining and the sections were mounted with Fluorescence Mounting Medium (DAKO). The sections were visualized on an Olympus FV1000MPE Confocal microscope and analyzed by FV10-ASW 4.06 software.

### Results

To assess the therapeutic potential for neonatal, potentially neurogenic microglia, we transplanted into cisterna magna, sorted neonatal and adult microglia into mice with symptomatic EAE and compared their effect on EAE progression. While transplanted adult microglia did not influence EAE progression, transplantation of neonatal microglia halted further EAE development and even resulted in improvement, manifesting with completely restored tail movement in several mice.

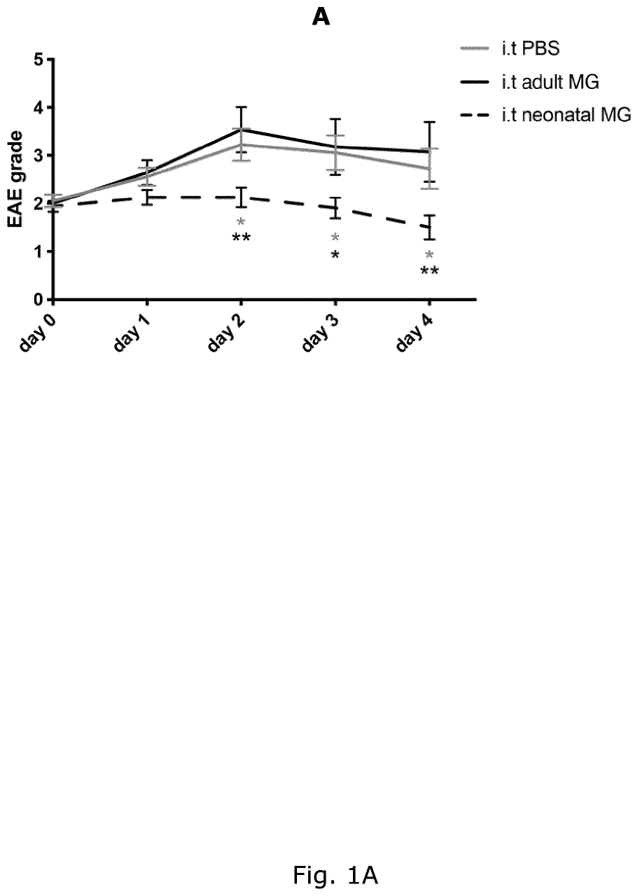

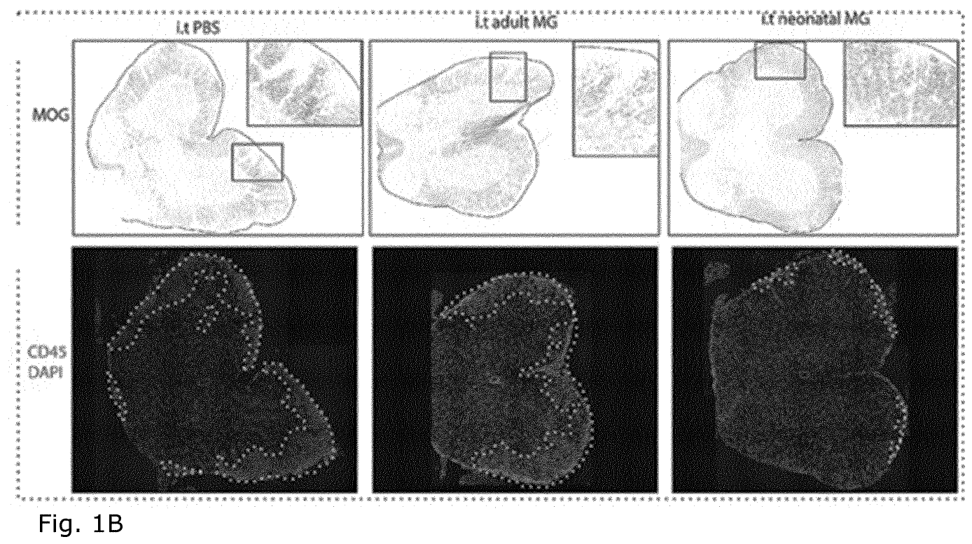

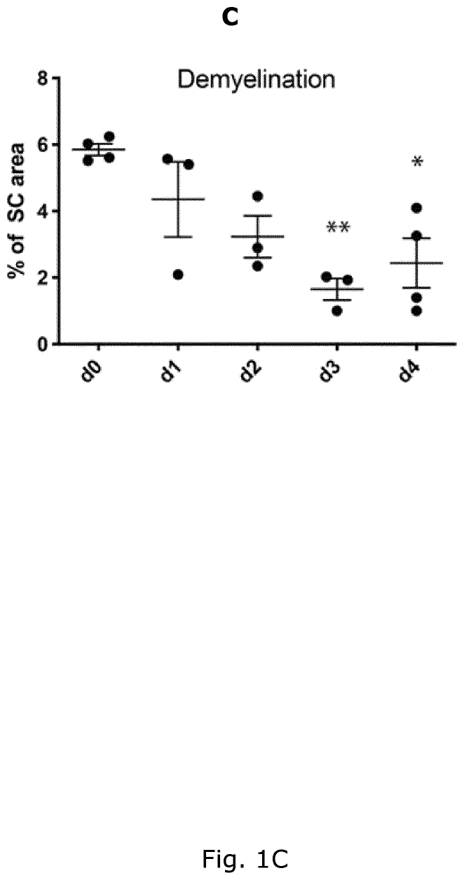

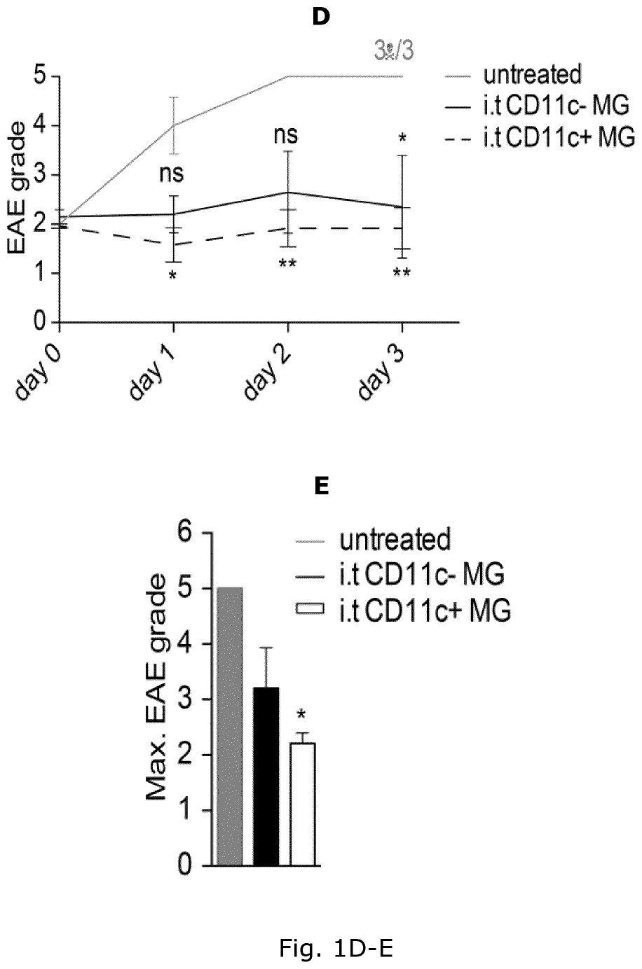

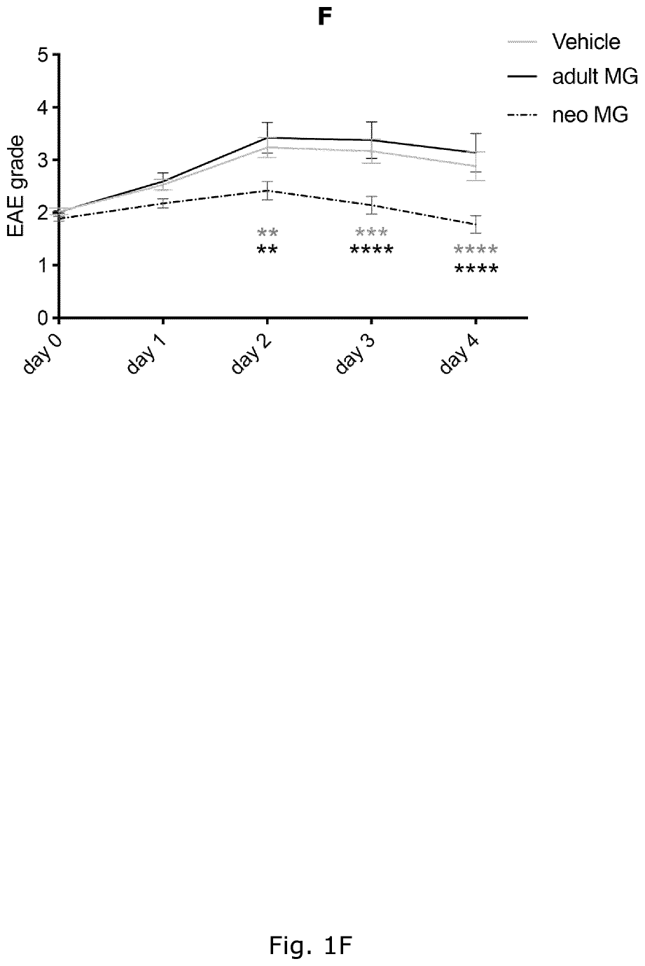

Unlike PBS-injected controls or mice receiving adult microglia, mice that received neonatal microglia never developed severe EAE and did not reach the ethical endpoint (FIG. 1A). Importantly, transplanted neonatal microglia were detectable within spinal cord lesions 2 days post transplantation (Data not shown). Spinal cords from EAE mice transplanted with neonatal microglia showed milder demyelination and less leukocyte infiltration (FIG. 1B) than adult microglia or PBS control groups. Moreover, the size of the demyelinated lesions was decreasing in time post transplantation suggesting that neonatal microglia induced active remyelination in opposed to suppression of demyelination (FIG. 1C). As CD11c+ subset of neonatal microglia has been strongly implicated in induction of primary myelination (9). We sorted CD11c⁺ and CD11c⁻ neonatal microglia and compared their effect on EAE progression. We observed significant amelioration of EAE symptoms caused by transplantation of CD11c⁺ neonatal microglia (FIG. 1D-E). Although CD11c− neonatal microglia also suppressed EAE, the effect was delayed. Importantly, no mice that received CD11c⁺ neonatal microglia developed severe EAE (1/6 mice reached max. grade 3) while 2/5 mice transplanted with CD11c⁻ neonatal microglia reached max. grade 5, and all the untreated mice reached the ethical endpoint.

### Conclusion

Transplantation of neonatal microglia leads to remyelination, immunomodulation and recovery from disability in animal model of MS. Hence, neonatal microglia transplantation leads to functional improvement in animal model of MS. Further, these results indicate that CD11c⁺ neonatal microglia have higher neuroprotective capacity than their CD11c⁻ counterparts.

### Example 2—Therapeutic Potential of Neonatal Microglia in Age-Related Cognitive Decline

### Aim of Study

To demonstrate therapeutic potential of neonatal microglia in age-related cognitive decline.

### Materials and Methods

### Mice

C57BL/6j bom female mice aged 7-8 weeks were obtained from Taconic Europe A/S, and maintained as a breeding colony in the Biomedical Laboratory, University of Southern Denmark (Odense) 24 ntil they reached 12 (young) or 79-83 weeks (aged) of age. 5xFAD mice were obtained from The Jackson Laboratory and maintained as a breeding colony in the Biomedical Laboratory, University of Southern Denmark (Odense) until reached 7 months of age. Neonatal mice (p3-p6) used for experiments were of mixed sex.

### CNS Tissue Dissociation

As in Example 1.

### Magnetic-Activated Cell Sorting (MACS-Sorting)—As in Example 2

Neontal microglia transplantation.

18-20 months old C57BL/6j bom female mice were randomized into 2 groups that received 3 consecutive injections of 10 μl PBS or 2×10⁵ neonatal microglia in PBS into cisterna magna.

7 months 5XFAD mice were randomized into 2 groups that received single injections of 10 μl PBS or 2×10⁵ neonatal microglia in PBS into cisterna magna.

### Barnes Maze

Mice were subjected to Barnes maze for assessment of short and long-term memory. Mice interacted with the Barnes maze in three phases: adaptation (1 day), training (3 days), and probe (1 day). On the adaptation day, mice were placed in the center of the maze inside a transparent starting cylinder for 30 s. Then mice were guided slowly by moving the cylinder towards the target hole that led to the escape box. After 10-15 s, the cylinder was removed and mice were given 2 min to independently enter through the target hole into the escape box. If they did not enter on their own during that time, they were gently guided. Mice were allowed to stay in the escape box for 2 min before being returned to a holding cage. In the training phase, mice were placed inside a non-transparent starting cylinder placed in the center of the maze for 15 s. At the end of the holding period, the aversive lights were turned on and they were allowed to explore the maze for 3 min. If a mouse found the escape box and entered it during that time, it was allowed to stay there for 1 min. If it did not find the escape box, it was gently guided towards it. The total number of trials each day was 4. On the probe day, the escape box was removed and the mice were placed inside the starting cylinder placed in the center of the maze for 15 s, the aversive lights were turned on, and the cylinder removed. Each mouse was given 1.5 min to explore the maze, thereafter the mouse was returned to its cage.

### Histology

Twelve-micrometer sections from 4% PFA-fixed, frozen brains of perfused mice were cut on a cryostat and stored in de Olmos cryoprotectant solution (17) at −14° C. or in −20° C. on Superfrost Plus slides (Thermo Scientific) respectively. Tissue sections were demasked in 70% formic acid diluted in H2O for 15 min at room temperature, followed by staining with biotinylated anti-amyloid beta (6E10, Biosite) antibody, which was detected by horse radish peroxidase-Streptavidin and developed in DAB solution and analyzed by ImageJ software.

### Results

To further assess neuroregenerative properties of neonatal microglia we performed three consecutive transplantations of sorted neonatal microglia into cisterna magna of aged mice and analysed their effect on short- and long-term memory outcome. As expected, aged animals showed short- and long-term memory deficits, presented as increased time needed to find escape box (primary latency) and increased number of errors made before finding the escape box in comparison to young animals.

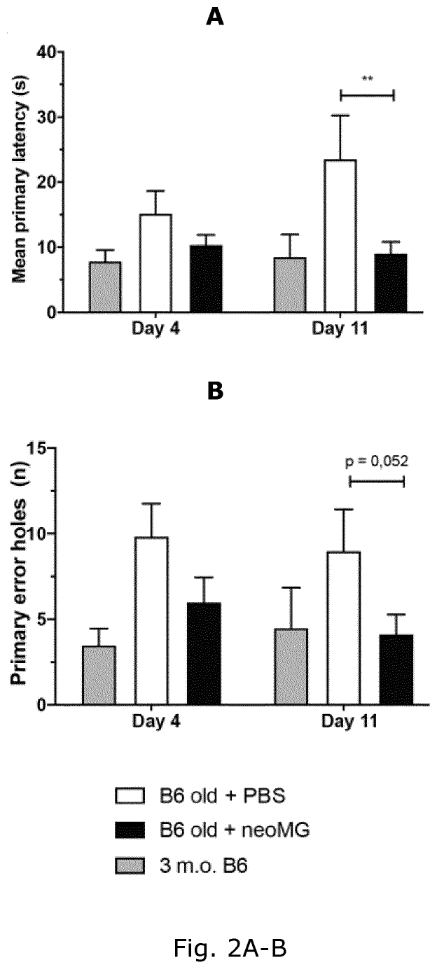

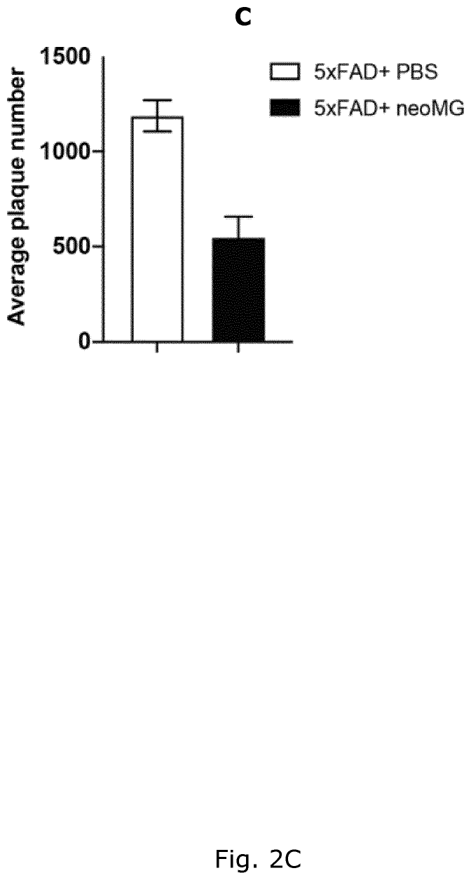

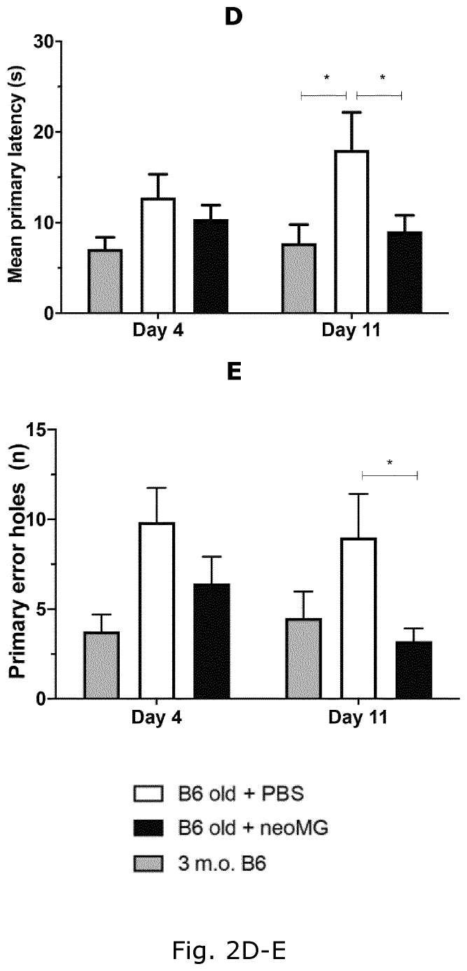

Interestingly, transplantation of neonatal microglia improved short-term memory, and led to restoration of long-term memory to the level seen in young animals (FIG. 2AB). This was even further improved when more data was included where it was found that transplantation of neonatal microglia restored short-term and long-term memory to the level seen in young animals (FIG. 2D-E).

In addition, we have shown that transplantation of neonatal microglia led to a substantial decrease of amyloid beta plaques in 5xFAD-animal model of Alzheimer's disease (FIG. 2C), suggesting a strong neuro-regenerative properties of neonatal microglia.

### Conclusion

Transplantation of neonatal microglia leads to improvement and even complete restoration of short-term memory and complete restoration of long-term memory. Hence, neonatal microglia transplantation leads to a restoration of the age-related memory loss.

### Example 3—Alternative Source of Neonatal Microglia-Like Cells

### Aim of Study

To identify clinically available source of neonatal microglia-like cells.

### Materials and Methods

### Mice

C57BL/6j bom female and male mice aged 7-8 weeks were obtained from Taconic Europe A/S, CX3CR1-GFP/GFP reporter mice were obtained from The Jackson Laboratory and maintained as a breeding colony in the Biomedical Laboratory, University of Southern Denmark (Odense). C57BL/6j bom female were crossed with CX3CR1-GFP/GFP males to generate CX3CR1-GFP/WT fetuses. CD45.1 mice were obtained from The Jackson Laboratory and maintained as a breeding colony in the Biomedical Laboratory, University of Southern Denmark (Odense). C57BL/6j bom (CD45.2) female were crossed with CD45.1 males to generate CD45.1/CD45.2 fetuses. The pregnancies were timed and pregnancy associated tissues were isolated at E17.5.

### Tissue Dissociation:

To isolate umbilical cords, placenta and YS, pregnant mice 17.5 days post coitus were anaesthetized with 200 mg/kg of pentobarbital and decapitated, tissues were isolated from fetuses and single cell suspensions were generated using Multi Tissue Dissociation Kit1 (Miltenyi Biotec). Erytrocytes were lysed from placental samples.

### Magnetic-Activated Cell Sorting (MACS-Sorting)

Cells were incubated for 15 min with CX3CR 1-biot (biolegend) followed by anti-biotin MicroBeads (Miltenyi Biotec), washed and sorted on manual MACS separator (Miltenyi Biotec) using MS column (Miltenyi Biotec). To ensure high purity the eluted cells were put through second MS column and eluted.

Fluorescence-Activated Cell Sorting (FACS-Sorting) for scRNAseq.

Placental cell suspension was incubated in blocking buffer (2% fetal bovine serum, Merck Life Science A/S, Søborg, Denmark; anti-Fc receptor CD16/CD32, BD Pharmingen; and 50 μg/mL Syrian Hamster IgG, Jackson ImmunoResearch Laboratories Inc., West Grove, PA, USA) before staining. anti-CD45.1 (Clone A20; BD Pharmingen) anti-CD45.2 (Clone 104; Biolegend) antibodies and live/dead marker (Sytox Blue Dead Cell Stain; Thermo Fisher Scientific). CD45.2+, CD45− maternal cells and CD45.1+, CD45.2+ fetal cells were sorted on a FACS Aria III (BD Biosciences).

### Flow Cytometry

Placenta, UC and YS cell suspensions were incubated in blocking buffer (2% fetal bovine serum, Merck Life Science A/S, Søborg, Denmark; anti-Fc receptor CD16/CD32, BD Pharmingen; and 50 μg/mL Syrian Hamster IgG, Jackson ImmunoResearch Laboratories Inc., West Grove, PA, USA) before staining. Staining was performed using anti-CD45 (Clone 30-F11; Biolegend), anti-Ly6C (Clone HK1.4; Biolegend), anti-F4/80 (Clone BM8; Biolegend), anti-CD117 (c-kit; Clone 2B8; Biolegend) antibodies and live/dead marker (Sytox Blue Dead Cell Stain; Thermo Fisher Scientific). Cell fluorescence data were acquired on a LSRII flow cytometer (BD Biosciences) and analyzed using FlowLogic software (Inivai).

### UC Cells Transplantation:

UC cells were isolated by MACS-sorting from CX3CR1GFP/WT fetuses and 2×10⁵ UC macrophages were transplanted to unmanipulated C57BL/6j bom female via stereotactic intrastriatal injection.

### Single-Cell RNA Library Preparation and Sequencing

Single-cell RNA library preparation and sequencing were performed by Amplexa Genetics A/S (Odense, Denmark) according to the 10× Genomics user guide. Approximately 15,500 not sure what you mean here cells/sample were mixed with 10× master mix reagents and loaded on a Chip B Single Cell (10× Genomics, Stockholm, Sweden) together with Single Cell 3′ v3 Gel Beads (10× Genomics) and partitioning oil to generate single cell Gel Beads-in-Emulsion (GEMs). The GEM generation procedure was controlled by a 10× Chromium Controller (10× Genomics). Then single cell reverse transcription took place at 53° C. in a standard thermal cycler, before the GEMs were broken using Recovery Agent (10× Genomics). The resulting first-strand cDNA was purified with DynaBeads MyOne Silane Beads (Thermo Fisher Scientific) and amplified by PCR using Single Cell 3′ GEM Kit v3.1. After a cDNA clean-up with SPRIselect Reagent Beads (Beckman Coulter), the cDNA concentrations were measured using Qubit dsDNA HS Assay Kit (Thermo Fisher Scientific). Hereafter next-generation sequencing libraries were constructed using Chromium Single Cell 3′ Library Kit v3. and Single Index Kit T. After a final double-sided fragment size selection, the fragment sizes and concentrations were measured using QIAxcel DNA High Resolution Kit (1200) (Qiagen) and KAPA Library Quantification Kit (Roche), respectively. Finally, the scRNA libraries were sequenced on an Illumina NovaSeq 6000 platform using NovaSeq 6000 S1 Reagent Kits (Read 1=28, 17 Index=8; Read 2=91).

### Single-Cell RNA-Sequencing Analysis

Raw data was processed using zUMIs (Parekh et al., 2018) mapping to mm10 and counting unique molecular identifiers (UMIs) assigned to exons of Ensemble transcripts (Ens79). Putative cell-containing droplets were identified using EmptyDrops (Lun et al., 2019) using a lower threshold of 100 UMIs.

Subsequently, high-quality cells were identified using Scater (McCarthy et al., 2017). Initially, outlier cells were removed when showing large median absolute deviations (≥3) in terms of number of detected genes, number of UMIs, percent of UMIs assigned to mitochondrial genes or percent of UMIs assigned to ribosomal genes. Lastly, the cells were filtered based on the number of detected genes (maternal≥500, fetal≥2000), the number of UMIs (maternal≥1000, fetal≥5000), the ratio between UMIs and genes (≤20). The genes were also filtered: all non-protein coding genes were removed. Regarding protein-coding genes, the transcript variant with the highest transcript level support was kept. Finally, only genes detected in at least one cell in either of the maternal datasets were included for maternal analysis and only genes detected in at least one cell in either of the fetal datasets were included for fetal analysis. After filtering, the datasets were normalized using Scran (Lun et al., 2016) and the two maternal and two fetal datasets were normalized to the same scale using Batchelor (Haghverdi et al., 2018).

For clustering, embedding and visualization, the two maternal datasets were merged into a single maternal dataset and two fetal datasets were merged into a single fetal dataset. Highly variable genes were identified using Seurat (Stuart et al., 2019) (maternal: 1500 genes, fetal: 5000), scaled and decomposed by principal component analysis (PCA). The first principal components (PCs) (maternal: 14, fetal: 20) were used for uniform manifold approximation and projection (UMAP) embedding. Initially, cells were coarsely clustered using Louvain clustering on the UMAP coordinate. Each coarse cluster was extracted, new highly variable genes (between 100 and 5000 genes) were identified, scaled, decomposed by PCA, embedded by UMAP (using 10 to 20 PCs) and clustered to achieve high resolution clustering by an approach similar to the hierarchal relationship between immune cell types and activation states. Differentially expressed genes between clusters or between conditions within clusters was identified using Seurat (Stuart et al., 2019).

### Histology

Twelve-micrometer sections from 4% PFA-fixed, frozen brains of perfused mice were cut on a cryostat and stored in de Olmos cryoprotectant solution (17) at −14° C. or in −20° C. on Superfrost Plus slides (Thermo Scientific) respectively.

Sections were washed in PBS and incubated for 10 min in ice-cold acetone. After repeated rinses with 0.2% Triton X100 in PBS (PBST) they were incubated for 1 h in 3% BSA in PBS to block unspecific binding. Next, sections were incubated overnight at 4° C. with corresponding primary antibodies: rabbit anti-IBA1 (Wako), Following primary antibody incubation, the sections were washed with PBST and incubated for 1 h with goat anti rabbit Alexa 594 (Invitrogen); After immunofluorescent labeling, nuclei were visualized by DAPI staining and the sections were mounted with Fluorescence Mounting Medium (DAKO). The sections were visualized on an Olympus FV1000MPE Confocal microscope and analyzed by FV 10-ASW 4.06 software.

### Results

As practical and ethical reasons limit the use of neonatal microglia in clinics, we aimed to assess alternative sources for such protective cells. Microglia develop from yolk sac primitive macrophages during embryonic development, and they are never replaced by fetal liver or bone-marrow progenitors. Taken the fate of microglia into consideration, ideally, the progenitors should be derived from YS originated macrophages and not bone-marrow cells to generate fully functional microglia-like cells. In contrast to mice, in human, the yolk sac is regressing during the course of pregnancy and is virtually absent at birth, thus cannot serve as a source for microglia progenitors, unless isolated from aborted fetuses. As during development these primitive macrophages migrate from the yolk sac to the brain, we hypothesized that some of them might still be found in other pregnancy associated tissues. To test that, we have analyzed murine placenta, umbilical cord tissue, amniotic fluid, and umbilical cord blood for presence of yolk sac derived primitive macrophages.

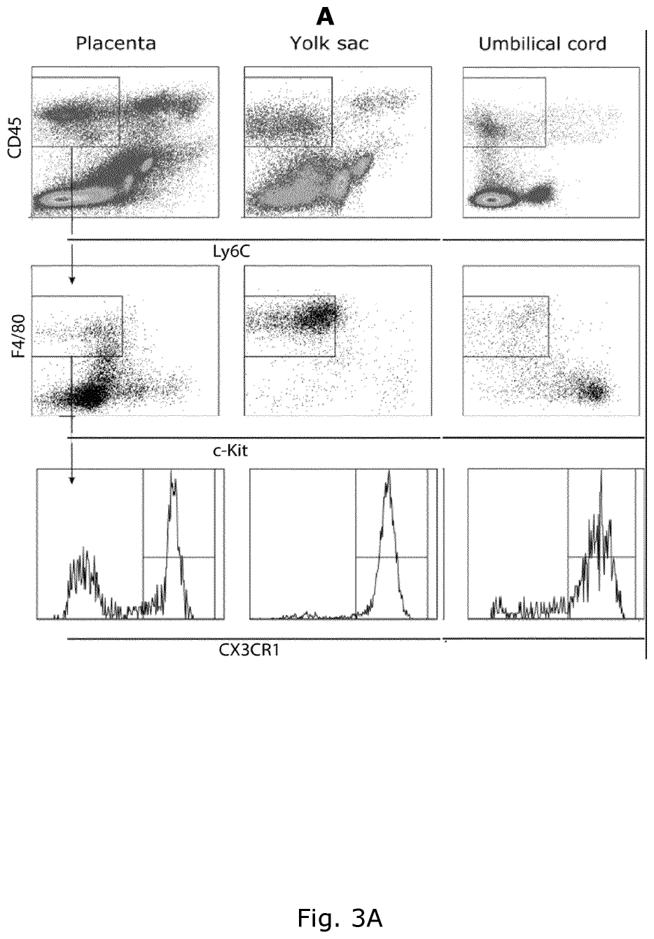

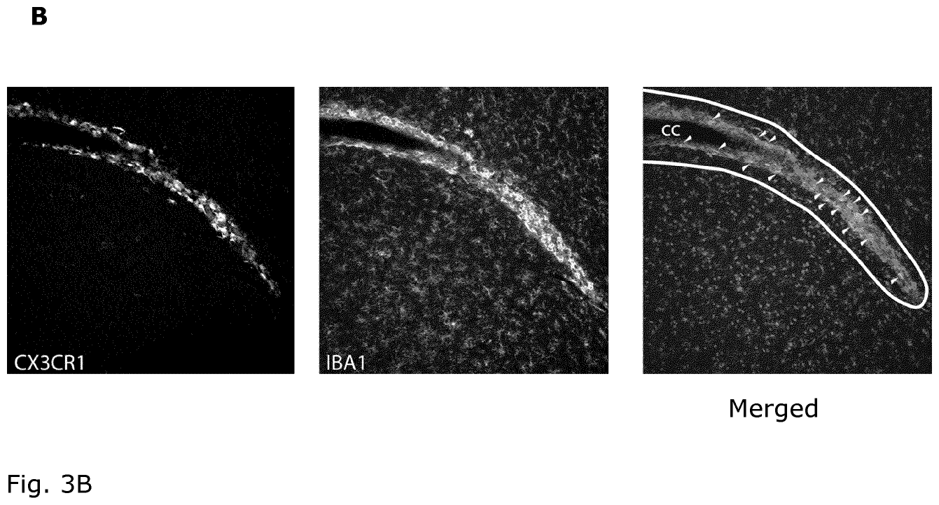

Interestingly, in mice we have discovered cells closely resembling yolk sac primitive macrophages (CD45+, CX3CR1+, F4/80+, c-kit−, Ly6C−) in all the tested tissues except for umbilical cord blood (FIG. 3A). Next, we performed histological analysis, to assess morphology and location of these cells within umbilical cord tissue and the placenta. Although, in umbilical cord tissue we observed morphologically and phenotypically homogenous CD45+ CX3CR1+ cell population throughout the tissue, in the placenta the fetal CD45+ CX3CR1+ cells were heterogenous (Data not shown). In order to understand this complexity, we performed scRNA seq analysis from murine placenta. As expected, the data showed high heterogeneity of the cells, showing 9 clusters of CX3CR1 positive cells (Data not shown). Interestingly, transcriptomes of the fetal cluster 7 and maternal cluster 4 significantly overlapped with CD11c+ neonatal microglia signature (Data not shown).

As a proof of concept, in further experiments we have used umbilical cord derived macrophages (UCM) as they are more homogenous and thus easier to isolate. To assess if UCM, can differentiate into microglia-like cells, we transplanted them into brain striatum and analyzed the brain tissues by confocal microscopy (FIG. 3B). Interestingly, we observed that the transplanted cells migrated away from the injection site to corpus callosum, they acquired microglia characteristic IBA1 expression as well as showed change in morphology (FIG. 3B), we have confirmed their survival up to at least 14 days post transplantation (Data not shown).

### Conclusion

Primitive macrophages can be isolated from umbilical cord and placenta. They, closely resemble yolk sac-derived microglia progenitors. When transplanted to the brain they acquire microglia characteristics such as expression of IBA1 and migratory potential. Hence, pregnancy associated tissues are a novel source for neonatal microglia-like cells.

### Example 4—Human Pregnancy Associated Tissues Contain Yolk Sac Primitive Macrophages'

### Aim of Study

To assess human pregnancy associated tissues as a source of neonatal microglia-like cells. Further, an aim is to establish a phenotype of such neonatal microglia-like cells in humans.

### Materials and Methods

### Human Material:

Human samples were acquired from caesarian section labored healthy patients.

A 5 cm piece of umbilical cord and chorionic plate of placenta was immediately after surgery, cut and placed in 4% PFA or sterile ice-cold PBS.

### Histology:

Four μm-thick serial sections were stained with anti-huCX3CR1 and counter-stained with hematoxylin. The stainings were performed on a Dako Autostainer Universal Staining System (Dako, Denmark A/S, Glostrup, Denmark).

### Tissue Dissociation:

5-10 cm pieces of umbilical cord and placental chorionic plate were isolated immediately after elective caesarian section deliveries and and transported in Ice-cold PBS. Single cell suspensions from placenta were generated using Multi Tissue Dissociation Kit1 (Miltenyi Biotec) and from umbilical cord using Human umbilical cord dissociation Kit (Miltenyi Biotec) with modification as follow: The vessels were flushed with ice-cold PBS, followed by vigorous shaking in PBS (changed few times) to remove blood, tissue was then cut in 0,5.1 cm pieces with scalpel and transferred to C-tube, Enzyme B was added to the mix for the last 15 min of dissociation. Erythrocytes were lysed from placental samples.

### Flow Cytometry

Placenta, UC cell suspensions were incubated in blocking buffer (2% fetal bovine serum, Merck Life Science A/S, Søborg, Denmark; and 50 μg/mL Syrian Hamster IgG, Jackson ImmunoResearch Laboratories Inc., West Grove, PA, USA) before staining. Staining was performed using biot-anti CX3CR1 (Biolegend), anti-CD45 (Biolegend), anti-CD14 (Biolegend), anti-CD11b (Biolegend), anti-CD117 (c-kit; Biolegend) antibodies and live/dead marker (Sytox green Dead Cell Stain; Thermo Fisher Scientific). Cell fluorescence data were acquired on a LSRII flow cytometer (BD Biosciences) and analyzed using FlowLogic software (Inivai).

### Results

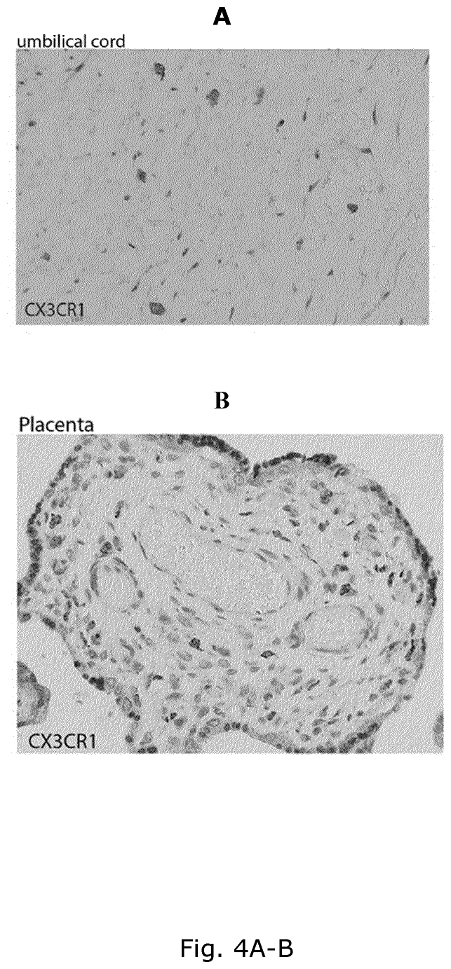

In order to assess if such primitive yolk sac derived macrophages are also present in human placenta and umbilical cord tissue, we have first performed histological stainings (FIG. 4A-B). Similar to murine tissues, we observed CX3CR1+ cells in chorionic plate (fetal part) of the placenta and in the Wharton's jelly of the umbilical cord of human tissue. Flow cytometry analysis showed similar phenotypical profile as observed in murine tissues, namely CX3CR1+, CD45+, CD11b+, CD14+/CD14−, c-kit−, and more detailed CX3CR1+, CD45+, CD11b+, CD14+/CD14−, c-kit−, CD47, and HLA-E+/− (Data not shown). Interestingly, it was observed that the cells may also be CD11b− and a later backtracking of mouse UCM transplanted cells showed that CD11b− cells were also identified in the tissues (data not shown).

### Conclusion

Primitive macrophages, resembling microglia progenitors can be isolated from human umbilical cord and placenta. The phenotype in humans was established to be CD45+, CD11b+, c-kit−, CD14+/CD14−, and CX3CR1+, and even more detailed to be CD45+, CD11b+/CD11b−, c-kit−, CD14+/CD14−, CD47, HLA-E+/− and CX3CR1+.

### Example 5—Therapeutic Potential of Umbilical Cord Derived Macrophages (UCM) in Alzheimer's Disease

### Aim of Study

To demonstrate the therapeutic potential of umbilical cord macrophages (UCM) in Alzheimer's Disease (AD).

### Materials and Methods

See also example 1.

### Mice

C57BL/6j bom female and male mice aged 7-8 weeks were obtained from Taconic Europe A/S and maintained as a breeding colony in the Biomedical Laboratory, University of Southern Denmark (Odense). The pregnancies were timed and umbilical crods were isolated at E17.5-E19. 5xFAD mice were obtained from The Jackson Laboratory and maintained as a breeding colony in the Biomedical Laboratory, University of Southern Denmark (Odense) until reached 7 months of age.

### Microglia Depletion

In order to transiently deplete resident microglia, mice were fed with PLX3397 (600 ppm) in diet gel for 7 days.

### Tissue Dissociation

To isolate umbilical cords pregnant mice 17.5-19 days post coitus were anaesthetized with 200 mg/kg of pentobarbital and decapitated, tissues were isolated from fetuses and single cell suspensions were generated using Multi Tissue Dissociation Kit1 (Miltenyi Biotec).

### Magnetic-Activated Cell Sorting (MACS-Sorting)

Cells were isolated by magnetic separation. Cells were incubated for 15 min with CX3CR1-biot (biolegend) followed by anti-biotin MicroBeads (Miltenyi Biotec), washed and sorted on manual MACS separator (Miltenyi Biotec) using MS column (Miltenyi Biotec). To ensure high purity the eluted cells were put through second MS column and eluted.

### Ucm Transplantation

UCM were isolated by MACS-sorting and 2×10⁵ cells were transplanted to unmanipulated 5XFAD females. 3 regimens were used:
1. UCM via stereotactic intracerebroventricular injection.
2. UCM+50ng MCSF via stereotactic intracerebroventricular injection (d0) followed by 50 ng MCSF via intacisternal injection (d1)
3. 50 ng MCSF via stereotactic intracerebroventricular injection (d0) followed by UCM via intacisternal injection (d1)

### Barnes Maze

Mice were subjected to Barnes maze for assessment of short and long-term memory. Mice interacted with the Barnes maze in three phases: adaptation (1 day), training (3 days), and probe (1 day). On the adaptation day, mice were placed in the center of the maze inside a transparent starting cylinder for 30 s. Then mice were guided slowly by moving the cylinder towards the target hole that led to the escape box. After 10-15 s, the cylinder was removed and mice were given 2 min to independently enter through the target hole into the escape box. If they did not enter on their own during that time, they were gently guided. Mice were allowed to stay in the escape box for 2 min before being returned to a holding cage. In the training phase, mice were placed inside a non-transparent starting cylinder placed in the center of the maze for 15 s. At the end of the holding period, the aversive lights were turned on and they were allowed to explore the maze for 3 min. If a mouse found the escape box and entered it during that time, it was allowed to stay there for 1 min. If it did not find the escape box, it was gently guided towards it. The total number of trials each day was 4. On the probe day, the escape box was removed and the mice were placed inside the starting cylinder placed in the center of the maze for 15 s, the aversive lights were turned on, and the cylinder removed. Each mouse was given 1.5 min to explore the maze, thereafter the mouse was returned to its cage.

### Phagocytosis Assay:

hUCM were isolated as described above, and cultured in vitro for 24 h, 37 C, in DMEM/F12, 10% FBS, 50 ng/ml rMCSF, after, the medium was removed, cells were washed and DMEM/F12 containing Beta-Amyloid (1-42), HiLyte™ Fluor 488-labeled was added to the cells and incubated for 4 h-24 h. The medium was then removed, cells were washed, detached from the plate and the cytospin was done attach the cells to a slide. The cells were counter-stained with DAPI to visualize the nuclei.

### Results

Transplantation of UCMs reduces AD symptoms in mice.

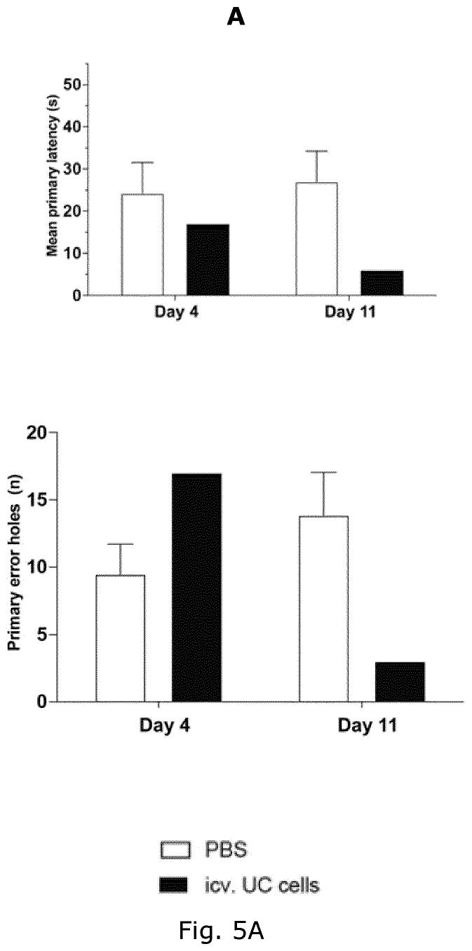

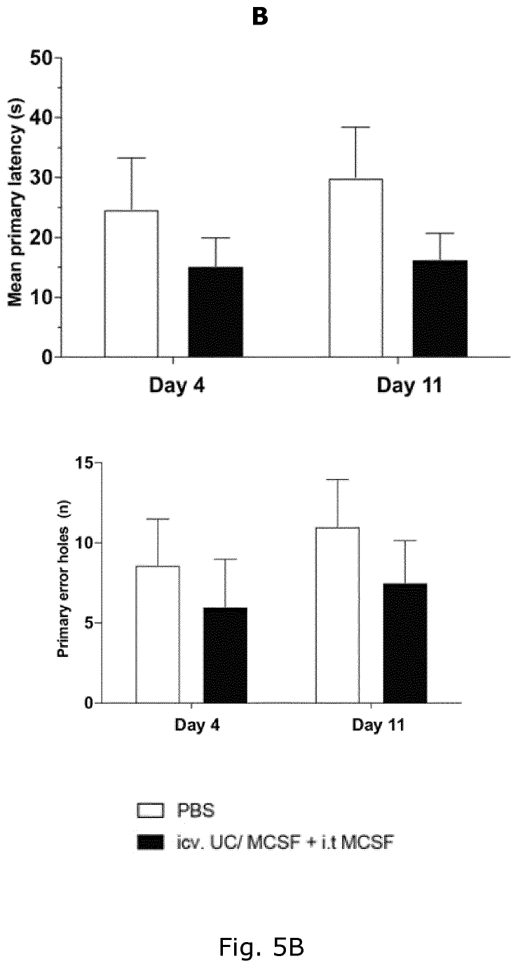

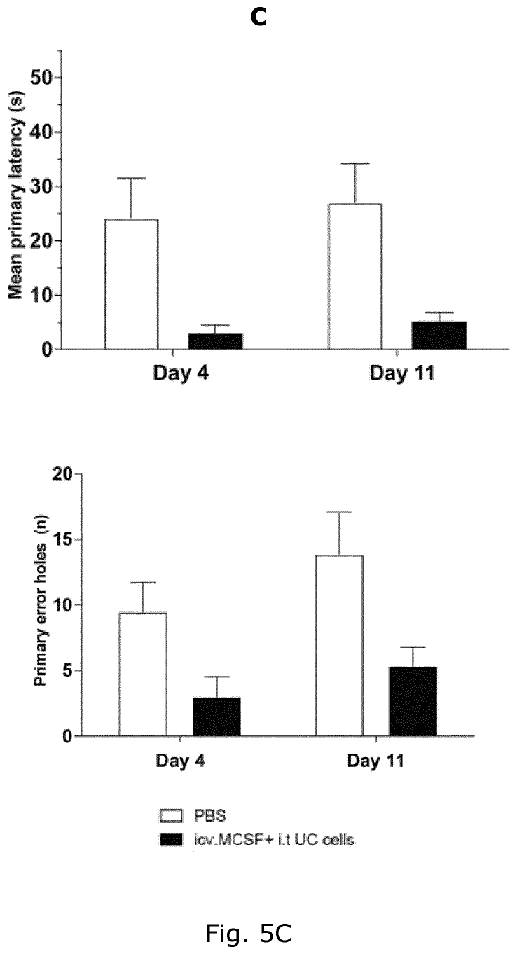

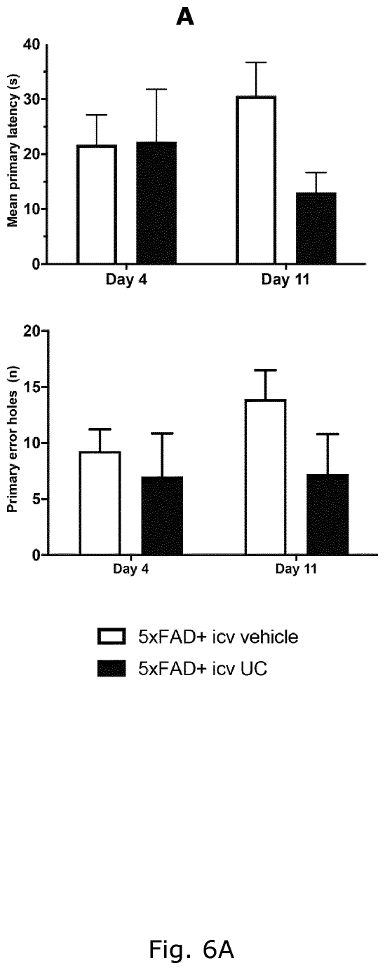

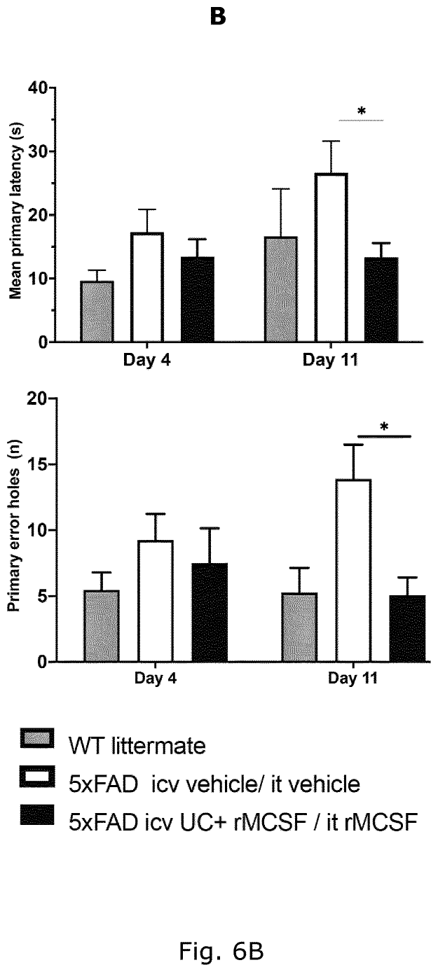

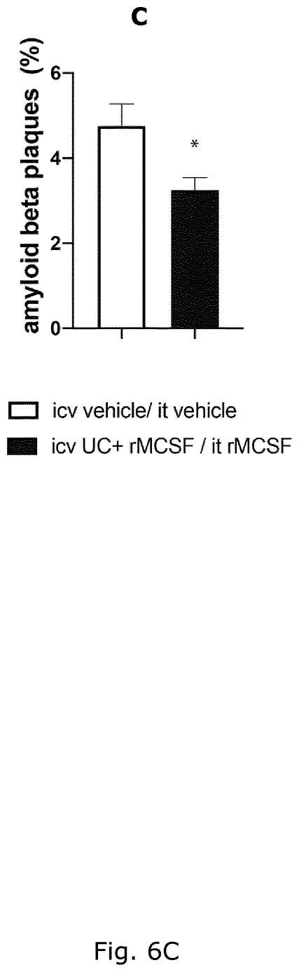

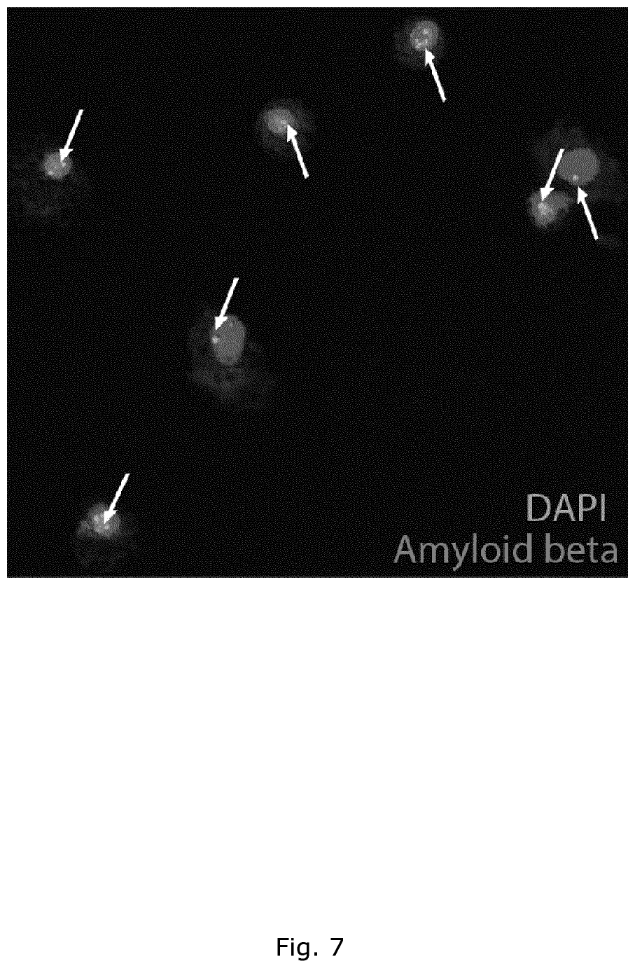

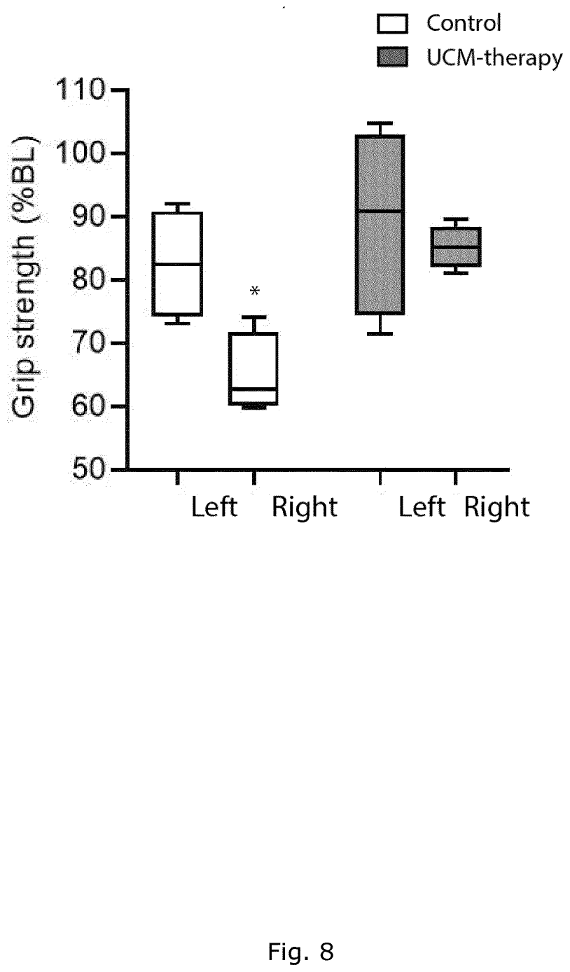

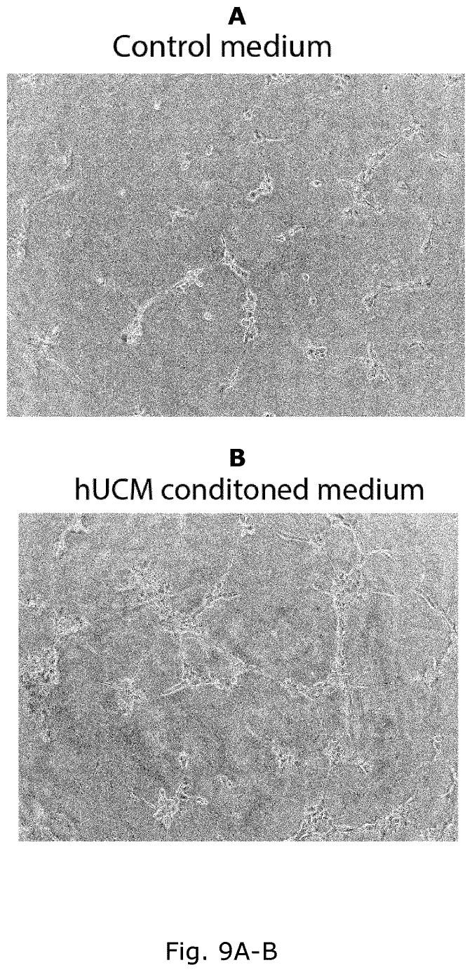

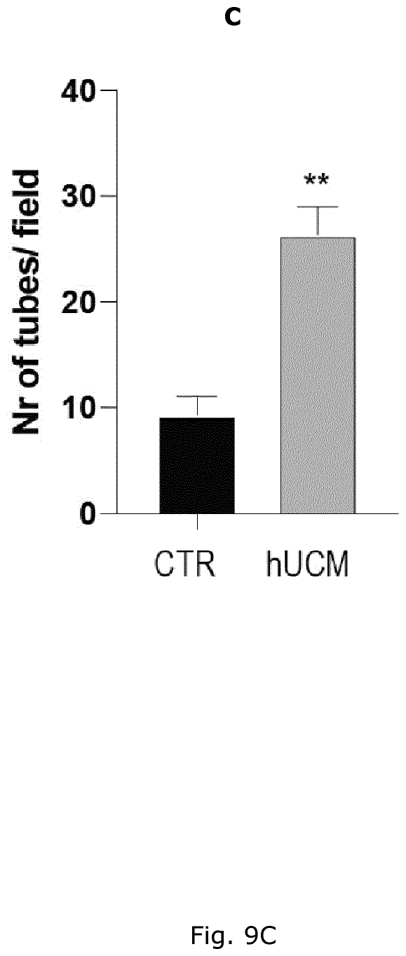

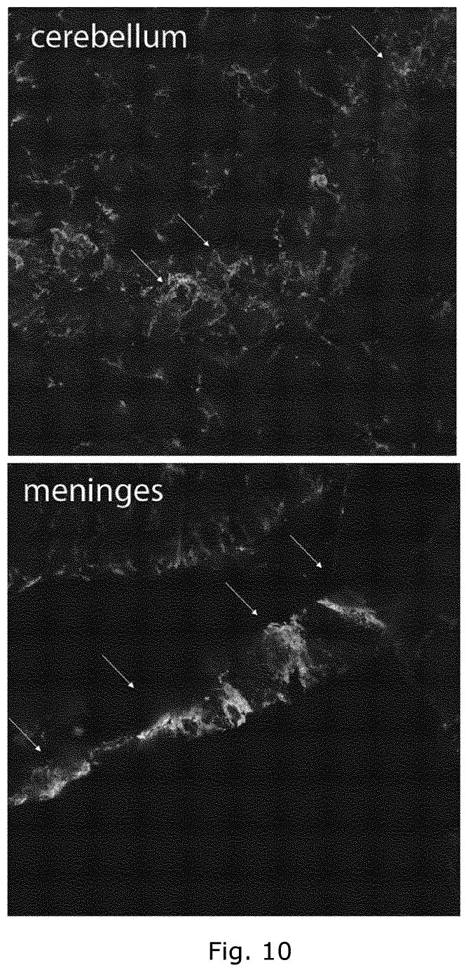

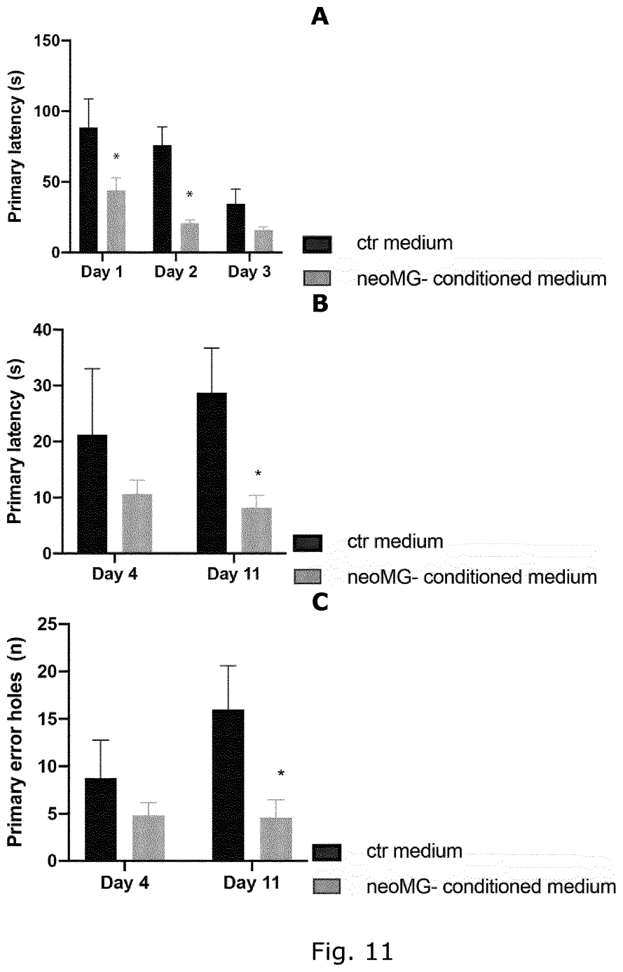

Having shown that UCM resemble yolk sac derived microglia progenitors (see example 3) and that they can differentiate into microglia we next assessed their therapeutic potential. As microglia, are heavily impaired in AD, we first depleted them by feeding the mice with diet gel containing CSF1R inhibitor, PLX3397 (600 ppi) as we previously described in (15). After the depletion, mice were fed for 4 days with a normal chow to clear up the PLX 3397 from the system, before transplantation of UCM. We chose the 4-day time point as there is already partial repopulation (ca. 20-0.40%) of resident microglia making it a perfect milieu for proliferation and differentiation of UCM into microglia. The cells were transplanted directly to the CSF via intracerebroventricular route (ICV). Interestingly, we observed that single UCM transplantation led to improvement in long-term but not in short-term memory (FIG. 5A, and FIG. 6A). In order to further facilitate differentiation into protective neonatal microglia-like cells the cells were supplemented with 50 ng MCSF (16) and ICV transplanted followed by injection of 50 ng MCSF into cisterna magna 1 day after cell transplantation (FIG. 5B, and FIG. 6B). Importantly this intervention led to restoration of short-and long-term memory to the level of wild type littermates, as well as led to significant reduction of amyloid beta in the brain (FIG. 6C). Alternatively, we injected 50 ng MCSF ICV followed by intracisternal cell transplantation a day after (FIG. 5C), also leading to therapeutic outcome that was not observed with injection of MCSF alone (not shown).

Phagocytic Capacity of hUCM.

Having shown, reduction of amyloid beta in vivo after the UCM transplantation we wanted to confirm such activity in cells isolated from human UC. We have incubated hUCM with amyloid beta and after 1 h and 4 h we could observe that virtually all cells were phagocytosis the amyloid beta (FIG. 7). Showing similar phagocytic properties as murine cells.

### Conclusion

Transplantation of UCM restores short- and long-term memory in animal model of AD and leads to reduction of amyloid beta plaques.

### Example 6—Therapeutic Potential of Umbilical Cord Derived Macrophages (UCM) in Stroke

### Aim of Study

To demonstrate the therapeutic potential of umbilical cord macrophages (UCM) in in stroke.

### Materials and Methods

See also example 1.

### Mice

C57BL/6j bom female and male mice aged 7-8 weeks were obtained from Taconic Europe A/S and maintained as a breeding colony in the Biomedical Laboratory, University of Southern Denmark (Odense). The pregnancies were timed and umbilical crods were isolated at E17.5-E19. pMCAO was induced in 8-12 weeks old female mice.

### Grip Strength Test:

Grip strength was assessed before (baseline) and five days post-surgery using a BIO-GT-3 grip strength meter (BIOSEB, Paris, France), which was connected to a stainless steel grid. The procedure involved horizontally pulling the mouse until it released its grip. The force exerted by the mouse was automatically detected and recorded. Measurements were taken for individual (right and left) and total (both) front paw strengths both before and after permanent middle cerebral artery occlusion (pMCAO). The results were expressed as grip strength, allowing for the calculation of asymmetry, which was determined by comparing changes in grip strength on day 5 relative to the baseline. Each mouse underwent five consecutive trials, with the highest force recorded being taken as the definitive score for that mouse.

### UCM Transplantation

UCM were isolated by MACS-sorting and 2×10⁵ cells were transplanted to unmanipulated 5XFAD females.

### Stroke Model:

Permanent left middle cerebral artery occlusion (pMCAO) was performed following the methods outlined by Gregersen et al. (2000) and Lambertsen et al. (2009). The procedure involved female mice, which were anesthetized using a mixture of Hypnorm (fentanyl citrate at 0.315 mg/ml and fluanisone at 10 mg/ml; VetaPharma Ltd, Leeds, UK), Midazolam (5 mg/ml midazolam hydrochloride; Hameln Pharma, Hameln, Germany), and distilled water in a 1:1:2 ratio. The surgical area, located between the orbit and the ear, was prepared by making a vertical incision with a scalpel. Subsequently, the temporal muscle was dissected, and a 1 mm hole was drilled into the skull to expose the middle cerebral artery (MCA). The left MCA was then permanently occluded using microbipolar coagulation. Post-operatively, the mice received sutures and were administered saline to prevent dehydration. They were then placed in a heating cabinet maintained at 28° C. for 24 hours to facilitate recovery. For post-surgical analgesia, Buprenorphine hydrochloride (0.001 mg/20 g, Temgesic) was administered three times at eight-hour intervals, starting from the time of surgery.”

hUCM Conditioned Medium:

hUCM were isolated as described above and cultured for 24 h in DMEM/F12, N2, Penstrep, 10% FBS, after the medium was removed, cells were washed and cultured for another 24 h in DMEM/F12, Penstrep. Conditioned medium was then collected and stored in −20 C for further use.

### In Vitro Angiogenesis Assay:

Flat bottom 96-well plates were coated with 50 μl cold growth factor-reduced Matrigel (Corning) using cold pipette and pipette tips. The plate was put in 37 oC for 30 min to solidify Matrigel. Meanwhile, human primary umbilical vein endothelial cells (HUVECs, ATCC) grown in Endothelial Cell Growth Medium (PromoCell) were collected by standard trypsinization. Cells were divided into separate Eppendorf tubes containing 50,000 cells, centrifuged and resuspended in appropriate media, after which they were seeded on the Matrigel-coated wells. The plate was observed after 4 h for tube formation and the images were taken under the light microscope.

### Results

Next, we explored the therapeutic potential of UCM in a mouse model of stroke. We employed a model of permanent middle cerebral artery occlusion (pMCAO) to induce stroke in mice¹⁸. Following this, either UCM or PBS was administered into the cisterna magna of the mice one day post-occlusion. The functional outcome was assessed by measuring grip strength asymmetry in the animals five days after the stroke event. Interestingly, while animals treated with PBS exhibited the expected stroke-induced grip strength asymmetry, this asymmetry was significantly reduced or even eliminated in animals treated with UCM, suggesting a notable improvement in motor function (FIG. 8).

Additionally, given the preliminary transcriptomic data hinting at the proangiogenic potential of UCM (data not shown), we further investigated its effects on angiogenesis in vitro. We demonstrated that conditioned medium derived from hUCM markedly enhanced endothelial cell tube formation in vitro, which is indicative of its angiogenic capability (FIG. 9A-C). This finding aligns with the hypothesized role of UCM in promoting angiogenesis, potentially contributing to its therapeutic effects observed in the stroke model.

### Conclusion:

These findings suggest that UCM transplantation can significantly improve motor function in a stroke model, potentially mediated by its proangiogenic properties as evidenced in vitro.

### Example 7—Regenerative Properties of Conditioned Medium from Neonatal Microglia

### Aim of Study

To study the regenerative potential of conditioned medium from neonatal microglia.

### Materials and Methods

### NeoMG Conditioned Medium:

Neonatal microglia were isolated as described above and cultured for 24 h in Dulbecco's Modified Eagle Medium: Nutrient Mixture F12 (DMEM/F12) (Gibco) media containing 10% FBS, 1× penicillin-streptomycin (Pen-Strep) and supplemented with 1× N-2, and 50 ng/ml interleukin 34 (IL34) and 50 ng/ml macrophage colony-stimulating factor (MCSF). Conditioned medium was then collected and stored in −20 C for further use.

24 h after seeding, media was replaced with fresh DMEM/F12 medium with 1× Pen-Strep. 24 h after media replacement, the supernatant was collected without the cells and frozen until use.

Control media (without cells) were subjected to identical manipulations.

Treatment with NeoMG-Conditioned Media

18-20 months old C57BL/6j bom female mice were randomized into 2 groups that received 3 consecutive injections of 10 μl control medium or NeoMG conditioned medium into cisterna magna.

### Results

In our study, we observed that neonatal microglia (neoMG), when transplanted into aged animals, predominantly localized in the meninges and, to a lesser extent, in the cerebellum (FIG. 10). This distribution led us to hypothesize that neoMG may secrete factors that contribute to neural regeneration, of note RNAseq data showed upregulation of factors such as osteopontin and IGF1. To investigate this, we prepared both conditioned media from the neoMG cultures and control media. These were administered intracisternally into the aged animals at intervals of every other day, for a total of three injections. We then assessed the animals'learning capabilities, short-term memory, and long-term memory. Our results demonstrated that the conditioned media from neoMG significantly enhanced learning abilities and memory in these animals, with a particularly notable improvement observed in long-term memory (FIG. 11).

### Conclusion

Conditioned medium can be generated from neoMG. The conditioned medium is capable of improving learning capabilities and restoring long-term memory in aged animals.

### REFERENCES

1. Hou Y, Dan X, Babbar M, Wei Y, Hasselbalch S G, Croteau D L, et al. Ageing as a risk factor for neurodegenerative disease. Nat Rev Neurol. 2019;15(10):565-81.
2. Collaborators GBDDF. Estimation of the global prevalence of dementia in 2019 and forecasted prevalence in 2050: an analysis for the Global Burden of Disease Study 2019. Lancet Public Health. 2022;7(2):e105-e25.
3. Huang L K, Chao S P, and Hu C J. Clinical trials of new drugs for Alzheimer disease. J Biomed Sci. 2020;27(1):18.
4. Hansen D V, Hanson J E, and Sheng M. Microglia in Alzheimer's disease. J Cell Biol. 2018;217(2):459-72.
5. Nimmerjahn A, Kirchhoff F, and Helmchen F. Resting microglial cells are highly dynamic surveillants of brain parenchyma in vivo. Science. 2005;308(5726):1314-8.
6. Paolicelli R C, Bolasco G, Pagani F, Maggi L, Scianni M, Panzanelli P, et al. Synaptic pruning by microglia is necessary for normal brain development. Science. 2011;333(6048):1456-8.
7. Kettenmann H, Kirchhoff F, and Verkhratsky A. Microglia: new roles for the synaptic stripper. Neuron. 2013;77(1):10-8.
8. Zhan Y, Paolicelli R C, Sforazzini F, Weinhard L, Bolasco G, Pagani F, et al. Deficient neuron-microglia signaling results in impaired functional brain connectivity and social behavior. Nature neuroscience. 2014;17(3):400-6.
9. Wlodarczyk A, Holtman I R, Krueger M, Yogev N, Bruttger J, Khorooshi R, et al. A novel microglial subset plays a key role in myelinogenesis in developing brain. EMBO J. 2017;36(22):3292-308.
10. Hagemeyer N, Hanft K M, Akriditou M A, Unger N, Park E S, Stanley E R, et al. Microglia contribute to normal myelinogenesis and to oligodendrocyte progenitor maintenance during adulthood. Acta Neuropathol. 2017;134(3):441-58.
11. Hamilton S P, and Rome L H. Stimulation of in vitro myelin synthesis by microglia. Glia. 1994;11(4):326-35.
12. Ueno M, Fujita Y, Tanaka T, Nakamura Y, Kikuta J, Ishii M, et al. Layer V cortical neurons require microglial support for survival during postnatal development. Nature neuroscience. 2013;16(5):543-51.
13. Ginhoux F, Greter M, Leboeuf M, Nandi S, See P, Gokhan S, et al. Fate mapping analysis reveals that adult microglia derive from primitive macrophages. Science. 2010;330(6005):841-5.
14. Mass E, Jacome-Galarza C E, Blank T, Lazarov T, Durham B H, Ozkaya N, et al. A somatic mutation in erythro-myeloid progenitors causes neurodegenerative disease. Nature. 2017;549(7672):389-93.
15. Wlodarczyk A, Khorooshi R, Marczynska J, Holtman I R, Burton M, Jensen K N, et al. Type I interferon-activated microglia are critical for neuromyelitis optica pathology. Glia. 2021;69(4):943-53.
16. Wlodarczyk A, Benmamar-Badel A, Cedile O, Jensen K N, Kramer I, Elsborg N B, et al. CSF1R Stimulation Promotes Increased Neuroprotection by CD11c+ Microglia in EAE. Frontiers in cellular neuroscience. 2018;12:523.
17. Khorooshi R, and Owens T. Detection and cellular localization of phospho-STAT2 in the central nervous system by immunohistochemical staining. Methods in molecular biology. 2013;967:179-88.
18 Clausen, B. H. et al. Interleukin-1beta and tumor necrosis factor-alpha are expressed by different subsets of microglia and macrophages after ischemic stroke in mice. Journal of neuroinflammation 5, 46, doi:10.1186/1742-2094-5-46 (2008).

### ITEMS

3. A composition comprising one or more microglia-like cells, such as neonatal microglia-like cells, wherein the one or more microglia-like cells has the phenotype CX3CR1+, CD11b+, CD45+, and cKit−.
2. The composition according to item 1, wherein the microglia-like cells are also CD11C+ and/or CD14−.
   3. The composition according to any of the preceding items, wherein the microglia-like cells are tissue-resident macrophages (TRM).
   3. The composition according to any of the preceding items, wherein the microglia-like cells are derived from umbilical cord tissue and/or placenta tissue.
   3. A composition comprising microglia-like cells isolated from umbilical cord tissue and/or comprising microglia-like cells isolated from placenta tissue.
6. The composition according to item 5, wherein the microglia-like cells have the phenotype CX3CR1+, CD11b+, CD45+, and cKit−.
7. A process for providing tissue-resident macrophages (TRM), the process comprising
   a) providing umbilical cord tissue and/or placenta tissue;
   b) purifying TRMs from said umbilical cord tissue and/or placenta tissue by selecting for cells that are CX3CR1+; and
   c) providing macrophages which are CX3CR1+.
8. The process according to item 7, wherein in step b), the cells are further selected for being CD45+, and/or CD14−, and/or cKit−; and/or wherein in step b), the cells are further selected for being CD11B+ and/or CD11C+.
9. The process according to any of items 7-8, wherein the provided cells are CX3CR1+; CD11b+, CD45+, and cKit−, preferably also being CD14−.
10. The process according to any of items 7-9, wherein the provided cells are cells as defined in any of items 1-6.
11. Cells obtained by or obtainable by a process according to any of items 7-10.
12. The composition according to any of items 1-6 or cells according to item 11, for use as a medicament.
13. The composition according to any of items 1-6 or cells according to item 11, for use in the treatment, alleviation and/or prevention of neurodegenerative diseases, demyelinating conditions, stroke and age-related cognitive decline.
14. The composition or cells for use according to any of items 12-13, wherein the composition or cells is administered by intrathecal delivery, such as intracisternal delivery, intracerebroventricular delivery, intracerebral delivery, intranasal delivery or by injection/delivery into the cisterna magna.
15. An in vitro method for determining the effect of at least one agent on a cell as defined in any of item 1-6 or item 11, the method comprising
   a) providing at least one cell as defined in any of item 1-6 or item 11;
   b) contacting the at least one cell from step a) with the biological agent; and
   c) determining the effect of the agent on the at least one cell.

1.-40. (canceled)

41. A method for the treatment, alleviation of symptoms, and/or prevention of neurodegenerative diseases, demyelinating conditions, cardiovascular diseases and/or age-related cognitive decline in a subject; the method comprising administering to the subject a composition comprising one or more microglia-like cells, wherein the one or more microglia-like cells has the phenotype CX3CR1+, CD45+, and cKit−.

42. The method according to claim 41, wherein the microglia-like cells are CD11b+.

43. The method according to claim 41, wherein the microglia-like cells are CD11C+ and CD14−.

44. The method according to claim 41, wherein the microglia-like cells are secreting osteopontin and/or IGF1.

45. The method according to claim 41, wherein the microglia-like cells are tissue-resident macrophages (TRM).

46. The method according to claim 41, wherein the microglia-like cells are derived from umbilical cord tissue.

47. The method according to claim 41, wherein the microglia-like cells are derived from placenta tissue.

48. The method according to claim 41, wherein the neurodegenerative disease is selected from the group consisting of Alzheimer's Disease, Parkinson's disease, multiple sclerosis (MS), and amyotrophic lateral sclerosis (ALS).

49. The method according to claim 41, wherein the demyelinating condition is selected from the group consisting of leukodystrophies, multiple sclerosis, spinal cord injury, peripheral nerve damage, Parkinson's disease, amyotrophic lateral sclerosis (ALS), and Alzheimer's disease.

50. The method according to claim 41, wherein the cardiovascular disease is selected from the group consisting of coronary artery disease, peripheral artery disease, cerebrovascular disease, ischemic stroke and hemorrhagic stroke.

51. The method according to claim 41, wherein the composition is administered by intrathecal delivery, intracerebroventricular delivery, intracerebral delivery, intranasal delivery or by delivery into the cisterna magna.

52. A process of providing a conditioned medium, the process comprising the steps:
a) providing a composition comprising one or more microglia-like cells, wherein the one or more microglia-like cells has the phenotype CX3CR1+, CD45+, and cKit−;
b) contacting a media with the composition of step a) for a time sufficient to condition the media; and
c) removing the cells from the media, thereby providing a conditioned media.

53. The process according to claim 52, wherein the microglia-like cells are CD11b+.

54. The process according to claim 52, wherein the provided conditioned media comprises secreted molecules from the microglia-like cells,

55. The process according to claim 52, wherein the provided conditioned media comprises one or more proteins selected from the group consisting of osteopontin and/or IGF1.

56. A method for the treatment, alleviation of symptoms, and/or prevention of neurodegenerative diseases, demyelinating conditions, cardiovascular diseases and/or age-related cognitive decline in a subject; the method comprising administering to the subject a conditioned media provided by the method according to claim 52.

57. The method according to claim 56, wherein the neurodegenerative disease is selected from the group consisting of Alzheimer's Disease, Parkinson's disease, multiple sclerosis (MS), and amyotrophic lateral sclerosis (ALS).

58. The method according to claim 56, wherein the demyelinating condition is selected from the group consisting of leukodystrophies, multiple sclerosis, spinal cord injury, peripheral nerve damage, Parkinson's disease, amyotrophic lateral sclerosis (ALS), and Alzheimer's disease.

59. The method according to claim 56, wherein the age-related cognitive decline is selected from the group consisting of decline of memory, decline of long-term memory, and/or decline of spatial memory.

60. The method according to claim 56, wherein cardiovascular diseases are selected from the group consisting of coronary artery disease, peripheral artery disease, cerebrovascular disease, ischemic stroke and hemorrhagic stroke.
